(19) United States
(12) Patent Application Publication
(10) Pub. No.: US 2025/0058133 A1
(43) Pub. Date: Feb. 20, 2025

(54) WEARABLE MEDICAL SYSTEM RESPONSIVE TO CERTAIN ECG PATTERNS

(71) Applicant: West Affum Holdings DAC, Dublin (IE)

(72) Inventor: Jaeho Kim, Redmond, WA (US)

(73) Assignee: West Affum Holdings DAC, Dublin (IE)

(21) Appl. No.: 18/497,246

(22) Filed: Oct. 30, 2023

Related U.S. Application Data
(60) Provisional application No. 63/533,558, filed on Aug. 18, 2023.

(51) Int. Cl. A61N 1/39; A61N 1/02; A61N 1/04

(52) U.S. Cl. CPC A61N 1/3925; A61N 1/025; A61N 1/046; A61N 1/0484; A61N 1/3904; A61N 1/3987

(57) ABSTRACT

A Wearable Medical System (WMS) that implements a wearable cardioverter defibrillator (WCD). In embodiments, the WMS detects when the patient's heart rate exceeds a high threshold heart rate. If it does, a present ECG peak amplitude is compared with a previous ECG peak amplitude that is stored in a memory. If there is deterioration enough to meet or exceed a peak amplitude decrease criterion, a therapeutic shock is administered to the patient. In additional optional embodiments, even if the peak amplitude decrease criterion is not met, it may be determined whether the ECG signal has QRS complexes that meet a morphology stability criterion. If yes, then VT may be detected, for which a therapeutic shock may be delivered upon confirmation. If not, then the event may be dismissed as electrical noise.

### CROSS-REFERENCE(S) TO RELATED APPLICATION(S)

This application claims the benefit of U.S. Provisional Application No. 63/533,558, filed on Aug. 18, 2023, the disclosure of which, as initially made, is hereby incorporated by reference in its entirety for all purposes.

### BACKGROUND

A wearable medical system (“WMS”) is an advanced form of a medical system. A WMS typically includes one or more wearable components that a patient can wear or carry, and possibly other components that can be portable, or stationary such as base station and/or an electric charger. The WMS may also include one or more associated software packages, such as software applications (“apps”), which can be hosted by the wearable component, and/or by a mobile device, and/or by a remote computer system that is accessible via a communications network such as the internet, and so on.

A WMS typically includes a sensor that can sense when a parameter of the patient is problematic, and cause the WMS to initiate an appropriate action. The appropriate action could be for the WMS to communicate with the patient or even with a bystander, to transmit an alert to a remotely located clinician, and to even administer treatment or therapy to the patient by itself. A WMS may actually include more than one sensor, which may sense more than one parameter of the patient. The multiple parameters may be used for determining whether or not to administer the treatment or therapy, or be suitable for detecting different problems and/or for administering respectively different treatments or therapies to the patient.

A WMS may also include the appropriate components for implementing a wearable cardioverter defibrillator (“WCD”), a pacer, and so on. Such a WMS can be for patients who have an increased risk of sudden cardiac arrest (“SCA”). In particular, when people suffer from some types of heart arrhythmias, the result may be that blood flow to various parts of the body is reduced. Some arrhythmias may result in SCA, which can lead to death very quickly, unless treated within a short time, such as 10 minutes. Some observers may have thought that SCA is the same as a heart attack, but it is not. For such patients, an external cardiac defibrillator can deliver a shock through the heart, and restore its normal rhythm. The problem is that it is hard for an external cardiac defibrillator to be brought to the patient within that short time. One solution, therefore, is for such patients to be given a WMS that implements a WCD. This solution is at least temporary and, after a while such as two months, the patient may instead receive a surgically implantable cardioverter defibrillator (“ICD”), which would then become a permanent solution.

A WMS that implements a WCD typically includes a harness, vest, belt, or other garment that the patient is to wear. The WMS system further includes additional components that are coupled to the harness, vest, or other garment. Alternately, these additional components may be adhered to the patient's skin by adhesive. These additional components include a unit that has a defibrillator, and sensing and therapy electrodes. When the patient wears this WMS, the sensing electrodes may make good electrical contact with the patient's skin and therefore can help sense the patient's Electrocardiogram (“ECG”). If the unit detects a shockable heart arrhythmia from the ECG, then the unit delivers an appropriate electric shock to the patient's body through the therapy electrodes. The shock can pass through the patient's heart and may restore its normal rhythm, thus saving their life.

A challenge for a WMS that implements a WCD is to find good algorithms for when to deliver a shock. Not all hearts are the same, and not all patient ECGs are the same. Another challenge is that sometimes the sensing electrodes rub against the skin of the patient, which injects electrical noise into the perceived ECG. The nature of such noise is often misdiagnosed as a heart rhythm that needs defibrillation, from tachycardia to VF. If the system tries to confirm that the patient is alive, the patient may start losing confidence in the system, and not wear it all the times.

All subject matter discussed in this Background section of this document is not necessarily prior art, and may not be presumed to be prior art simply because it is presented in this Background section. Plus, any reference to any prior art in this description is not, and should not be taken as, an acknowledgement or any form of suggestion that such prior art forms parts of the common general knowledge in any art in any country. Along these lines, any recognition of problems in the prior art discussed in this Background section or associated with such subject matter should not be treated as prior art, unless expressly stated to be prior art. Rather, the discussion of any subject matter in this Background section should be treated as part of the approach taken towards the particular problem by the inventor(s). This approach in and of itself may also be inventive.

### SUMMARY

The present description gives instances of Wearable Medical Systems (WMSs), storage media that may store programs, and methods, the use of which may help overcome problems and limitations of the prior art.

A Wearable Medical System (WMS) that implements a wearable cardioverter defibrillator (WCD). In embodiments, the WMS detects when the patient's heart rate exceeds a high threshold heart rate. If it does, a present ECG peak amplitude is compared with a previous ECG peak amplitude that is stored in a memory. If there is deterioration enough to meet or exceed a peak amplitude decrease criterion, a therapeutic shock is administered to the patient.

An advantage and/or benefit can be that a good criterion is thus implemented for the WCD to know when to deliver a shock. In addition, this criterion is computationally quick to achieve.

In additional optional embodiments, even if the peak amplitude decrease criterion is not met, it may be determined whether the ECG signal has QRS complexes that meet a morphology stability criterion. If yes, then VT or SVT may be detected. If VT has been detected, a shock may be delivered upon confirmation. If not, then the event may be dismissed as electrical noise.

An advantage and/or benefit of the additional optional embodiments can be that more instances can be ruled out when electrical noise is misdiagnosed as tachycardia. This way a false alarm is less likely to be issued to the patient, and therefore preserve the patient's faith and confidence in the system.

As such, it will be appreciated that embodiments have utility, and in fact may cause results that are larger than the sum of their individual parts.

These and other features and advantages of the claimed invention will become more readily apparent in view of the embodiments described and illustrated in this specification, namely in this written specification and the associated drawings.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a diagram of sample components of a wearable medical system (“WMS”) that implements a wearable cardioverter defibrillator (“WCD”), and which is made according to embodiments.

FIG. 2A is a diagram showing a view of the inside of a sample garment embodiment that can be a support structure of a WMS that implements a WCD, such as that of FIG. 1.

FIG. 2B is a diagram showing a view of the outside of the sample garment of FIG. 2A.

FIG. 2C is a diagram showing a front view of how the sample garment of FIGS. 2A and 2B is intended to be worn by a patient.

FIG. 2D is a diagram showing a back view of how the sample garment of FIGS. 2A and 2B is intended to be worn by a patient.

FIG. 3 is a diagram showing a partial front view of another patient wearing a sample garment embodiment of an alternate style as worn by a patient, and which can be a support structure of a WMS that implements a WCD such as that of FIG. 1.

FIG. 4 is a diagram showing sample embodiments of electronic components of a WMS that implements a WCD, and which can be used with the garment of FIG. 2A or of FIG. 3.

FIG. 5 is a diagram showing sample components of a unit of FIG. 1, which is made according to embodiments.

FIG. 6 is a conceptual diagram for illustrating an example how multiple electrodes may be used for sensing ECG signals along different vectors in a WMS that implements a WCD according to embodiments.

FIG. 7 is a time diagram of a segment of a waveform amplitude of an ideal ECG signal, further indicating aspects that can be monitored by embodiments.

FIG. 8 is a block diagram showing sample contents of a memory of FIG. 5 according to embodiments.

FIG. 9 is a mind map for describing how to treat an event where a patient heart rate exceeds a high threshold heart rate, according to embodiments.

FIG. 10 is a mind map for describing an optional refinement of FIG. 9 according to additional embodiments, and in which a sudden onset event has been detected.

FIG. 11 is a mind map for describing the optional refinement of FIG. 10 according to the additional embodiments, but in which a sudden onset event has not been detected.

FIG. 12 shows diagrams of sample steps for computing an ECG morphology parameter according to embodiments.

FIG. 13 shows a sample equation for how a morphology parameter may be computed in embodiments.

FIG. 14 shows a sample ECG signal during a heart's normal sinus rhythm (NSR), and the ECG morphology parameter of Organization computed according to the equation of FIG. 13.

FIG. 15 shows a sample ECG signal during a heart's Ventricular Fibrillation (VF), and the ECG morphology parameter of Organization computed according to the equation of FIG. 13.

FIG. 16 is a flowchart for illustrating sample methods according to embodiments.

FIG. 17 is a mind map for describing how to cancel a decided-up shock delivery, according to further embodiments.

FIG. 18 is a flowchart for illustrating sample methods according to the further embodiments.

### DETAILED DESCRIPTION

As has been mentioned, the present description is about wearable medical systems (WMS) that implement wearable cardioverter defibrillators (WCD). Embodiments are now described in more detail.

A wearable medical system (“WMS”) that implements a wearable cardioverter defibrillator (“WCD”) according to embodiments may protect an ambulatory patient by electrically restarting their heart if needed. Such a WMS may have a number of components. These components can be provided separately as modules that can be interconnected, or can be combined with other components, and so on. Examples are now described.

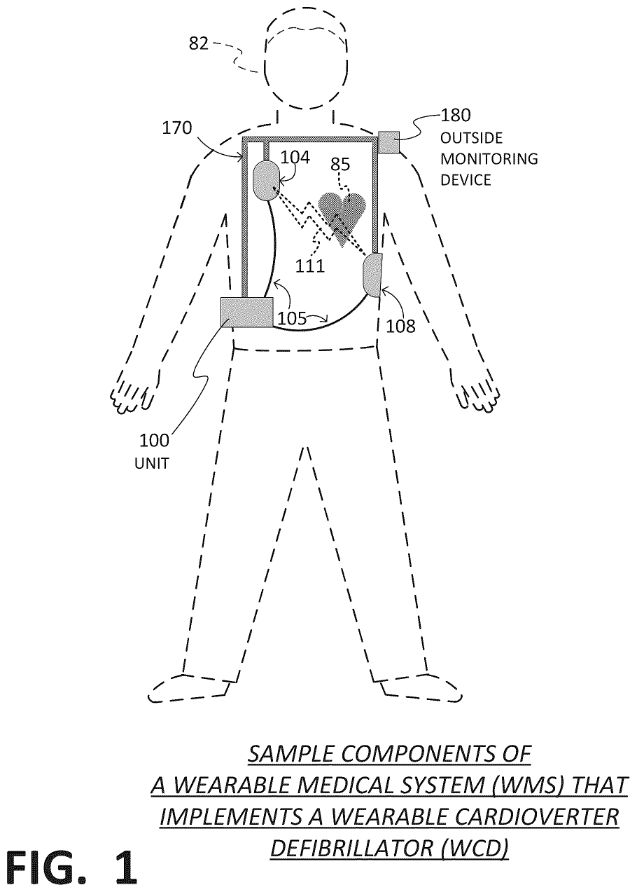

FIG. 1 depicts a patient 82. The patient 82 may also be referred to as the person 82 and/or wearer 82, since the patient 82 is wearing components of the WMS. The patient 82 is ambulatory, which means that, while wearing the wearable component(s) of the WMS, the patient 82 can walk around, be in a vehicle, and so on. In other words, the patient 82 is not necessarily bed-ridden. While the patient 82 may be considered to be also a “user” of the WMS, this definition is not exclusive to the patient 82. For instance, a user of the WMS may also be a clinician such as a doctor, nurse, emergency medical technician (EMT), or other similarly tasked and/or empowered individual or group of individuals. In some cases, a user may even be a bystander. The particular context of these and other related terms within this description should be interpreted accordingly.

A WMS that implements a WCD according to embodiments can be configured to defibrillate the patient who is wearing the designated components of the WMS. Defibrillating can be by the WMS delivering an electrical charge to the patient's body in the form of an electric shock. The electric shock can be delivered in one or more pulses.

In particular, FIG. 1 also depicts components of a WMS that implements a WCD and is made according to embodiments. One such component is a support structure 170 that is wearable by the ambulatory patient 82. Accordingly, the support structure 170 can be configured to be worn by the ambulatory patient 82 for at least several hours per day, and also during the night. That, for at least several days, and maybe even a few months. It will be understood that the support structure 170 is shown only generically in FIG. 1, and in fact partly conceptually. FIG. 1 is provided merely to illustrate concepts about the support structure 170, and is not to be construed as limiting how the support structure 170 is implemented, or how it is worn.

The support structure 170 can be implemented in many different ways. For example, it can be implemented in a single component or a combination of multiple components. In embodiments, the support structure 170 could include a vest, a half-vest, a garment, etc. In such embodiments such items can be worn similarly to analogous articles of clothing. In embodiments, the support structure 170 could include a harness, one or more belts or straps, etc. In such embodiments, such items can be worn by the patient around the torso, hips, over the shoulder, etc. In embodiments, the support structure 170 can include a container or housing, which can even be waterproof. In such embodiments, the support structure can be worn by being attached to the patient's body by adhesive material, for example as shown and described in U.S. Pat. No. 8,024,037. The support structure 170 can even be implemented as described for the support structure of US Pat. App. No. US2017/0056682, which is incorporated herein by reference. Of course, in such embodiments, the person skilled in the art will recognize that additional components of the WMS can be in the housing of a support structure instead of being attached externally to the support structure, for example as described in the US2017/0056682 document. There can be other examples.

The embodiments of FIG. 1 include a sample unit 100. In embodiments, the unit 100 is sometimes called a main electronics module. In embodiments, the unit 100 implements an external defibrillator. In embodiments, the unit 100 implements an external pacer instead of, or in addition to, an external defibrillator. In embodiments that include a pacer, the WMS may detect when the patient's heart rhythm slows down or when the patient has asystole, and the pacer may pace to increase the heart rate. In such embodiments, the WMS may pace the patient first, and hopefully not have to resort to the full intervention of defibrillation. Of course, if the patient does not respond to the pacing and their heart rhythm deteriorates further, the WMS may then later cause one or more defibrillation shocks to be delivered.

The embodiments of FIG. 1 also include sample therapy electrodes 104, 108, which are electrically coupled to unit 100 via electrode leads 105. The therapy electrodes 104, 108 are also called defibrillation electrodes or just electrodes. The therapy electrodes 104, 108 can be configured to be worn by the patient 82 in a number of ways. For instance, the unit 100 and the therapy electrodes 104, 108 can be coupled to the support structure 170, directly or indirectly. In other words, the support structure 170 can be configured to be worn by the ambulatory patient 82 so as to maintain at least one of the therapy electrodes 104, 108 on the body of the ambulatory patient 82, while the patient 82 is moving around, etc. The therapy electrodes 104, 108 can be thus maintained on the body by being attached to the skin of the patient 82, simply pressed against the skin directly or through garments, etc. In some embodiments the therapy electrodes 104, 108 are not necessarily pressed against the skin, but become biased that way upon sensing a condition that could merit intervention by the WMS. In addition, many of the components of the unit 100 can be considered coupled to the support structure 170 directly, or indirectly via at least one of the therapy electrodes 104, 108.

When the therapy electrodes 104, 108 make good electrical contact with the body of the patient 82, the unit 100 can administer, via the therapy electrodes 104, 108, a brief, strong electric pulse 111 through the body. The pulse 111 is also known as defibrillation pulse, shock, defibrillation shock, therapy, electrotherapy, therapy shock, discharge, discharging, etc. The pulse 111 is intended to go through and restart the heart 85, in an effort to save the life of the patient 82. The defibrillation pulse 111 can have an energy suitable for its purpose, such as at least 100 Joule (“J”), 200 J, 300 J, and so on. For pacer embodiments, the pulse 111 could alternately be depicting a pacing pulse. At least some of the stored electrical charge can be caused to be discharged via at least two of the therapy electrodes 104, 108 through the ambulatory patient 82, so as to deliver to the ambulatory patient 82 a pacing sequence of pacing pulses. The pacing pulses may be periodic, and thus define a pacing period and the pacing rate. There is no requirement, however, that the pacing pulses be exactly periodic. A pacing pulse can have an energy suitable for its purpose, such as at most 10 J, 5 J, usually about 2 J, and so on. The pacer therefore is delivering current to the heart to start a heartbeat. In either case, the pulse 111 has a waveform suitable for this purpose.

A prior art defibrillator typically decides whether to defibrillate or not based on an ECG signal of the patient. However, the unit 100 may initiate defibrillation, or hold-off defibrillation, based on a variety of inputs, with the ECG signal merely being one of these inputs.

A WMS that implements a WCD according to embodiments can collect data about one or more parameters of the patient 82. For collecting such data, the WMS may optionally include at least an outside monitoring device 180. The device 180 is called an “outside” device because it could be provided as a standalone device, for example not within the housing of the unit 100. The device 180 can be configured to sense or monitor at least one local parameter. A local parameter can be a parameter of the patient 82, or a parameter of the WMS, or a parameter of the environment, as described later in this document.

For some of these parameters, the device 180 may include one or more sensors or transducers. Each one of such sensors can be configured to sense a parameter of the patient 82, or of the environment, and to render an input responsive to the sensed parameter. In some embodiments the input is quantitative, such as values of a sensed parameter; in other embodiments the input is qualitative, such as informing whether or not a threshold is crossed, and so on. Such inputs about the patient 82 are also called physiological inputs and patient inputs. In embodiments, a sensor can be construed more broadly, as encompassing more than one individual sensors.

Optionally, the device 180 is physically coupled to the support structure 170. In addition, the device 180 may be communicatively coupled with other components that are coupled to the support structure 170, such as with the unit 100. Such communication can be implemented by the device 180 itself having a communication module, as will be deemed applicable by a person skilled in the art in view of this description.

A WMS that implements a WCD according to embodiments preferably includes sensing electrodes, which can sense an ECG of the patient. In embodiments, the device 180 stands for such sensing electrodes. In those embodiments, the sensed parameter of the patient 82 is the ECG of the patient, the rendered input can be time values of a waveform of the ECG signal, and so on.

In embodiments, one or more of the components of the shown WMS may be customized for the patient 82. This customization may include a number of aspects. For instance, the support structure 170 can be fitted to the body of the patient 82. For another instance, baseline physiological parameters of the patient 82 can be measured for various scenarios, such as when the patient is lying down (various orientations), sitting, standing, walking, running, and so on. These baseline physiological parameters can be the heart rate of the patient 82, motion detector outputs, one for each scenario, etc. The measured values of such baseline physiological parameters can be used to customize the WMS, in order to make its diagnoses more accurate, since patients' bodies differ from one another. Of course, such parameter values can be stored in a memory of the WMS, and so on. Moreover, a programming interface can be made according to embodiments, which receives such measured values of baseline physiological parameters. Such a programming interface may input automatically these in the WMS, along with other data.

The support structure 170 is configured to be worn by the ambulatory patient 82 so as to maintain the therapy electrodes 104, 108 on a body of the patient 82. As mentioned before, the support structure 170 can be advantageously implemented by clothing or one or more garments. Such clothing or garments do not have the function of covering a person's body as a regular clothing or garments do, but the terms “clothing” and “garment” are used in this art for certain components of the WMS intended to be worn on the human body in the same way as clothing and garments are. In fact, such clothing and garments of a WMS can be of different sizes for different patients, and even be custom-fitted around the human body. And, regular clothing can often be worn over portions or all of the support structure 170. Examples of the support structure 170 are now described.

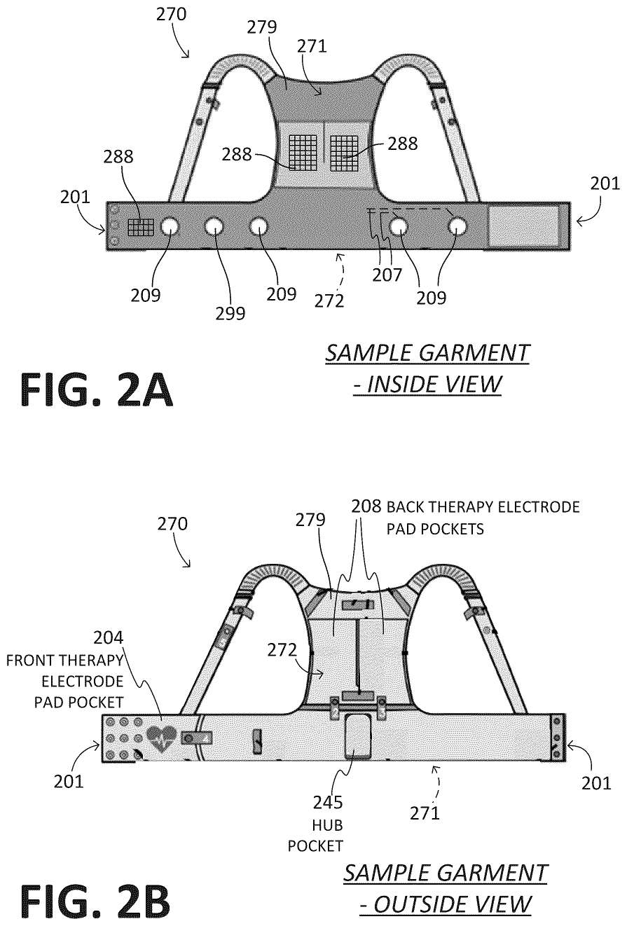

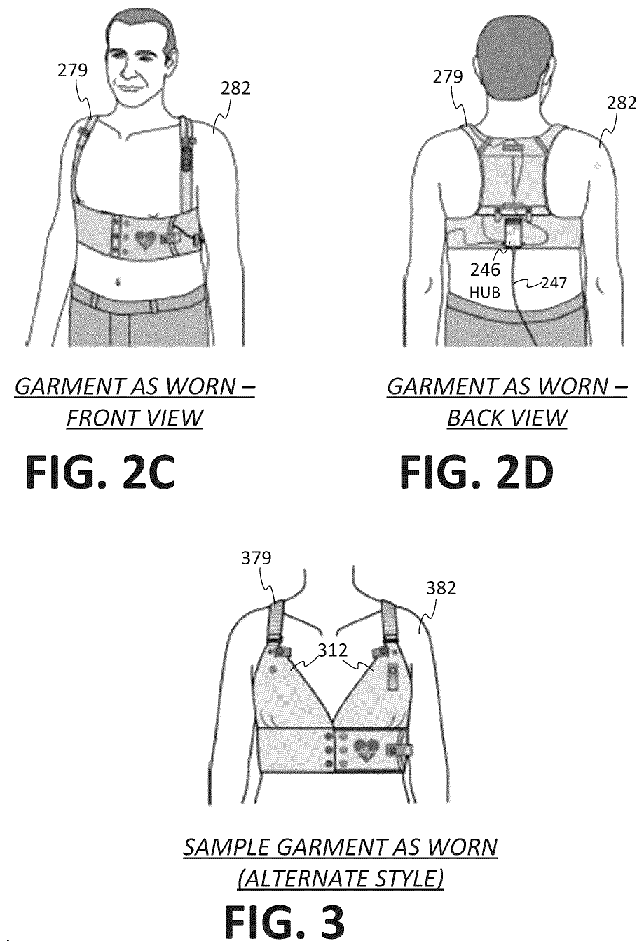

FIG. 2A shows a support structure 270 of a WMS that implements a WCD, such as the support structure 170 of FIG. 1. The support structure 270 is implemented by a vest-like wearable garment 279 that is shown flat, as if placed on a table. The inside side 271 of the garment 279 is seen as one looks at the diagram from the top, and it is the side contacting the body of the wearer when the garment 279 is worn. The outside side 272 of the garment 279 is opposite the inside side 271. To be worn, tips 201 can be brought together while surrounding the torso, and affixed to each other, either at their edges or partly overlapping. Appropriate mechanisms can hold together the tips 201, such as hooks and loops, Velcro® material, and so on.

The garment 279 can be made of suitable combinations of materials, such as fabric, linen, plastic, and so on. In places, the garment 279 can have two adjacent surfaces for defining between them pockets for the pads of the electrodes, for enclosing the leads or wires of the electrodes, and so on. Moreover, in FIG. 2A one can see meshes 288 which are the interior side of pockets accessible from the outside. The meshes can be made from flexible material such as loose netting, and so on.

ECG signals in a WMS that implements a WCD may sometimes include too much electrical noise for analyzing the ECG signal. To ameliorate the problem, multiple ECG sensing electrodes are provided in embodiments. These multiple ECG sensing electrodes define different vectors for sensing ECG signals along different ECG channels. These different ECG channels therefore present alternative options for analyzing the patient's ECG signal. The patient impedance along each ECG channel may also be sensed, and thus be part of the patient input.

In the example of FIG. 2A, multiple ECG sensing electrodes 209 are provided, which can be seen protruding from the inside surface of the garment 279. These ECG sensing electrodes 209 can be affixed to the inside surface of the garment 279, while their leads or wires 207 can be located mostly or completely within the garment 279. These ECG sensing electrodes 209 are intended to contact the skin of the person when the garment 279 is worn, and can be made from suitable material for good electrical contact. Such a material can be a metal, such as silver. An additional ECG-sensing electrode 299 may play the role of a Right Leg Drive (“RLD”) in the ECG analysis. In this context “RLD” is a custom electrical term, and embodiments do not require that the electrode 299 be actually placed on a leg of the patient.

FIG. 2B shows the outside side 272 of the garment 279. One can appreciate that pockets are included that are accessible from the outside, such as a hub pocket 245. In addition a pocket 204 is provided for a front therapy electrode pad, plus two pockets 208 are provided for two back therapy electrode pads. The pads of the therapy electrodes can be placed in the pockets 204, 208, and contact the skin of the patient through the respective meshes 288 that were seen in FIG. 2A. The electrical contact can be facilitated by conductive fluid that can be deployed in the area, when the time comes for a shock.

FIG. 2C is a diagram showing a front view of how the garment 279 would be worn by a patient 282. It will be appreciated that the previously described ECG sensing electrodes 209, 299 of FIG. 2A are maintained against the body of the patient 282 from the inside side of the garment 279, and thus are not visible in FIG. 2C.

FIG. 2D is a diagram showing the back view of FIG. 2C. A hub 246 has been placed in the hub pocket 245 that is shown in FIG. 2B. A cable 247 emerges from the hub 246, which can be coupled with a unit for the system, as described later in this document.

FIGS. 2A-2D do not show any physical support for a unit such as the unit 100 of FIG. 1. In these embodiments, such a unit may be carried in a purse, on a belt, by a strap over the shoulder, or additionally by further adapting the garment 279, and so on.

FIG. 3 is a diagram showing a partial front view of another patient 382 wearing another garment 379. The garment 379 is of an alternate style than the garment 279, in that it further includes breast support receptacles 312, as was described for instance in U.S. Pat. No. 10,926,080. This style of garment may be more comfortable if the patient 382 is a woman.

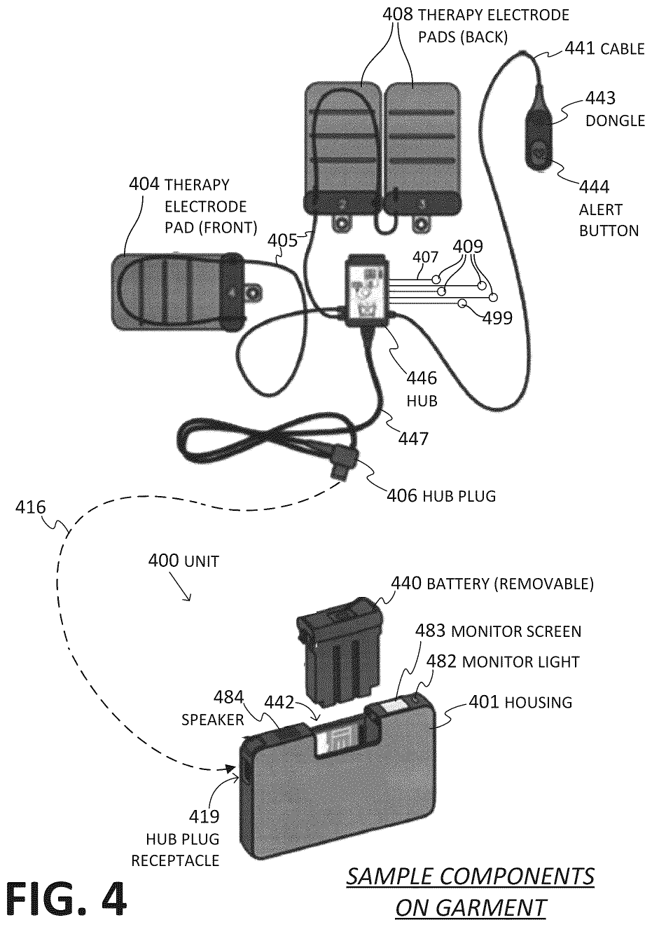

FIG. 4 shows sample electronic components that can be used with the garments 279, 379. The components of FIG. 4 include a unit 400, shown at the lower portion of FIG. 4. The unit 400 includes a housing 401, and a hub plug receptacle 419 at the housing 401.

The unit 400 includes a battery opening 442 at the housing 401. The battery opening 442 is configured to receive a removable battery 440. A system according to embodiments can have two identical such batteries 440, one plugged into the housing 401 while another one (not shown) is being charged by a charger (not shown). The batteries can then be interchanged when needed.

The unit 400 also includes devices for implementing a user interface. In this example, these devices include a monitor light 482, a monitor screen 483 and a speaker 484. Additional devices may include a vibrating mechanism, and so on.

The unit 400 can implement many of the functions of the unit 100 of FIG. 1. In the embodiment of FIG. 4, however, some of the functions of the unit 100 are implemented instead by a separate hub 446, which can be connected to the unit 400. The hub 446 is smaller and lighter than the unit 400, and can accommodate multiple electrical connections to other components of FIG. 4. A cable 447, similar to the cable 247 of FIG. 2D, emerges from the hub 446 and terminates in a hub plug 406. The hub plug 406 can be plugged into the hub plug receptacle 419 of the unit 400 according to an arrow 416.

ECG sensing electrodes 409, 499, plus their wires or leads 407 are further shown conceptually in FIG. 4 for completeness. The wires or leads 407 that can be configured to be coupled to the hub 446.

The components of FIG. 4 also include the therapy electrode pads 404, 408. The therapy electrode pad 404 can be inserted into the pocket 204 of FIG. 2B, while the therapy electrode pads 408 can be inserted into the pockets 208 of FIG. 2B. The shock is generated between the therapy electrode pad 404 and the therapy electrode pads 408 taken together. Indeed, the therapy electrode pads 408 are electrically connected to each other. The therapy electrode pads 404, 408, have leads 405, which can be configured to be coupled to the hub 446.

The components of FIG. 4 further include a dongle 443 with an alert button 444. The dongle 443 can be configured to be coupled to the hub 446 via a cable 441. The alert button 444 can be used by the patient to give emergency input to the WMS. For instance, the alert button 444 can be used by the patient to notify the system that the patient is actually alive and an imminent shock is not actually needed, which may otherwise happen in the event of a false positive detection of a shockable heart rhythm of the patient.

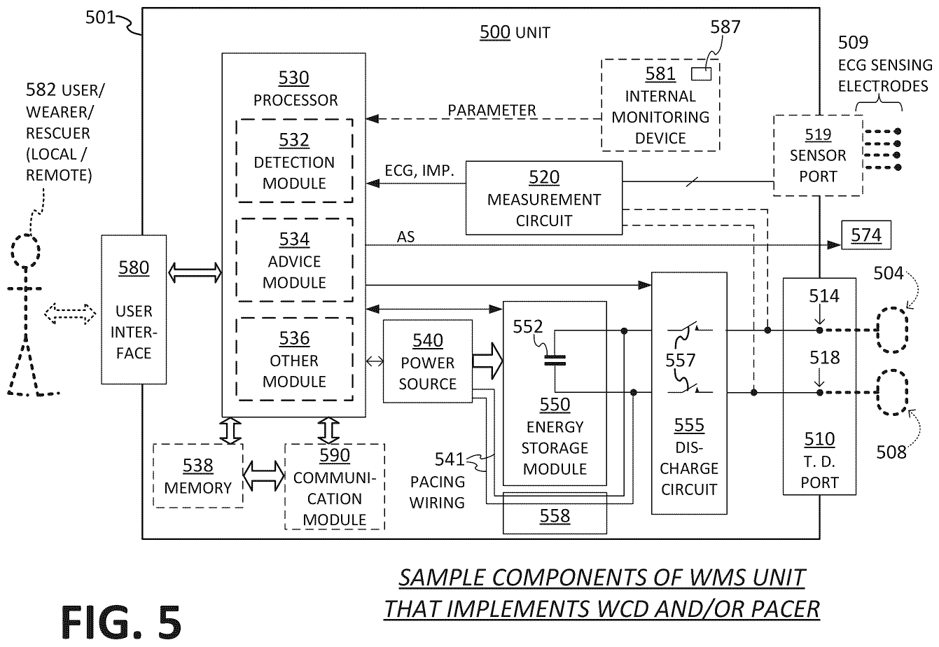

FIG. 5 shows a sample unit 500, which could be the unit 100 of FIG. 1. The unit 500 implements an external defibrillator and/or a pacer. The sample unit 500 thus combines the functions of the unit 400 and of the hub 446 of FIG. 4. The components shown in FIG. 5 can be provided in a housing 501, which may also be referred to as casing 501.

The unit 500 may include a user interface (UI) 580 for a user 582. User 582 can be the patient 82, also known as patient 582, also known as the wearer 582. Or, the user 582 can be a local rescuer at the scene, such as a bystander who might offer assistance, or a trained person. Or, the user 582 might be a remotely located trained caregiver in communication with the WMS, such as a clinician.

The user interface 580 can be made in a number of ways. The user interface 580 may include output devices, which can be visual, audible or tactile, for communicating to a user by outputting images, sounds or vibrations. Images, sounds, vibrations, and anything that can be perceived by user 582 can also be called human-perceptible indications. As such, an output device according to embodiments can be configured to output a human-perceptible indication (HPI). Such HPIs can be used to alert the patient, sound alarms that may be intended also for bystanders, and so on. There are many instances of output devices. For example, an output device can be a light that can be turned on and off, a screen to display what is sensed, detected and/or measured, and provide visual feedback to the local rescuer 582 for their resuscitation attempts, and so on. Another output device can be a speaker, which can be configured to issue voice prompts, alerts, beeps, loud alarm sounds and/or words, and so on. These can also be for bystanders, when defibrillating or just pacing, and so on. Examples of output devices were the monitor light 482, the monitor screen 483 and the speaker 484 of the unit 400 seen in FIG. 4.

The user interface 580 may further include input devices for receiving inputs from users. Such users can be the patient 82, 582, perhaps a local trained caregiver or a bystander, and so on. Such input devices may include various controls, such as pushbuttons, keyboards, touchscreens, one or more microphones, and so on. An input device can be a cancel switch, which is sometimes called an “I am alive” switch or “live man” switch. In some embodiments, actuating the cancel switch can prevent the impending delivery of a shock, or of pacing pulses. In particular, in some embodiments a speaker of the WMS is configured to output a warning prompt prior to an impending or planned defibrillation shock or a pacing sequence of pacing pulses being caused to be delivered, and the cancel switch is configured to be actuated by the ambulatory patient 82 in response to the warning prompt being output. In such embodiments, the impending or planned defibrillation shock or pacing sequence of the pacing pulses is not caused to be delivered. An example of a cancel switch was the alert button 444 seen in FIG. 4.

The unit 500 may include an internal monitoring device 581. The device 581 is called an “internal” device because it is incorporated within the housing 501. The monitoring device 581 can sense or monitor patient parameters such as patient physiological parameters, system parameters and/or environmental parameters, all of which can be called patient data. In other words, the internal monitoring device 581 can be complementary of, or an alternative to, the outside monitoring device 180 of FIG. 1. Allocating which of the parameters are to be monitored by which of the monitoring devices 180, 581 can be done according to design considerations. The device 581 may include one or more sensors, as also described elsewhere in this document.

Patient parameters may include patient physiological parameters. Patient physiological parameters may include, for example and without limitation, those physiological parameters that can be of any help in detecting by the WMS whether or not the patient is in need of a shock or other intervention or assistance. Patient physiological parameters may also optionally include the patient's medical history, event history and so on. Examples of such parameters include the above-described electrodes to detect the ECG, blood oxygen level, blood flow, blood pressure, blood perfusion, pulsatile change in light transmission or reflection properties of perfused tissue, heart sounds, heart wall motion, breathing sounds and pulse. Accordingly, the monitoring devices 180, 581 may include one or more sensors or transducers configured to acquire patient physiological signals. Examples of such sensors and transducers include one or more electrodes to detect ECG data, a perfusion sensor, a pulse oximeter, a device for detecting blood flow (e.g. a Doppler device), a sensor for detecting blood pressure (e.g. a cuff), an optical sensor, illumination detectors and sensors perhaps working together with light sources for detecting color change in tissue, a motion sensor, a device that can detect heart wall movement, a sound sensor, a device with a microphone, an SpO2 sensor, and so on. In view of this disclosure, it will be appreciated that such sensors can help detect the patient's pulse, and can therefore also be called pulse detection sensors, pulse sensors, and pulse rate sensors. In addition, a person skilled in the art may implement other ways of performing pulse detection.

In some embodiments, the local parameter reflects a trend that can be detected in a monitored physiological parameter of the patient 82, 582. Such a trend can be detected by comparing values of parameters at different times over short and long terms. Parameters whose detected trends can particularly help a cardiac rehabilitation program include: a) cardiac function (e.g. ejection fraction, stroke volume, cardiac output, etc.); b) heart rate variability at rest or during exercise; c) heart rate profile during exercise and measurement of activity vigor, such as from the profile of an accelerometer signal and informed from adaptive rate pacemaker technology; d) heart rate trending; e) perfusion, such as from SpO2, CO2, or other parameters such as those mentioned above, f) respiratory function, respiratory rate, etc.; g) motion, level of activity; and so on. Once a trend is detected, it can be stored and/or reported via a communication link, along perhaps with a warning if warranted. From the report, a physician monitoring the progress of the patient 82, 582 will know about a condition that is either not improving or deteriorating.

Patient state parameters include recorded aspects of the patient 582, such as motion, posture, whether they have spoken recently plus maybe also what they said, and so on, plus optionally the history of these parameters. Or, one of these monitoring devices could include a location sensor such as a Global Positioning System (GPS) location sensor. Such a sensor can detect the location, plus a speed of the patient can be detected as a rate of change of location over time. Many motion detectors output a motion signal that is indicative of the motion of the detector, and thus of the patient's body. Patient state parameters can be very helpful in narrowing down the determination of whether SCA is indeed taking place.

A WMS made according to embodiments may thus include a motion detector. In embodiments, a motion detector can be implemented within the outside monitoring device 180 or within the internal monitoring device 581. A motion detector of a WMS according to embodiments can be configured to detect a motion event. A motion event can be defined as is convenient, for example a change in posture or motion from a baseline posture or motion, etc. In such cases, a sensed patient parameter is motion. Such a motion detector can be made in many ways as is known in the art, for example by using an accelerometer and so on. In this example, a motion detector 587 is implemented within the monitoring device 581.

System parameters of a WMS can include system identification, battery status, system date and time, reports of self-testing, records of data entered, records of episodes and intervention, and so on. In response to the detected motion event, the motion detector may render or generate, from the detected motion event or motion, a motion detection input that can be received by a subsequent device or functionality.

Environmental parameters can include ambient temperature and pressure. Moreover, a humidity sensor may provide information as to whether or not it is likely raining. Presumed patient location could also be considered an environmental parameter. The patient location could be presumed, if the monitoring device 180 or 581 includes a GPS location sensor as per the above, and if it is presumed or sensed that the patient is wearing the WMS.

The unit 500 includes a therapy delivery port 510 and a sensor port 519 in the housing 501. In contrast, in FIG. 4 these ports are located at the hub 446.

In FIG. 5, the therapy delivery port 510 can be a socket in the housing 501, or other equivalent structure. The therapy delivery port 510 includes electrical nodes 514, 518. Therapy electrodes 504, 508 are shown, which can be as the therapy electrodes 104, 108. Leads of the therapy electrodes 504, 508, such as the leads 105 of FIG. 1, can be plugged into the therapy delivery port 510, so as to make electrical contact with the nodes 514, 518, respectively. It is also possible that the therapy electrodes 504, 508 are connected continuously to the therapy delivery port 510, instead. Either way, the therapy delivery port 510 can be used for guiding, via electrodes, to the wearer at least some of the electrical charge that has been stored in an energy storage module 550 that is described more fully later in this document. When thus guided, the electric charge will cause the shock 111 to be delivered.

The sensor port 519 is also in the housing 501, and is also sometimes known as an ECG port. The sensor port 519 can be adapted for plugging in the leads of ECG sensing electrodes 509. The ECG sensing electrodes 509 can be as the ECG sensing electrodes 209. The ECG sensing electrodes 509 in this example are distinct from the therapy electrodes 504, 508. It is also possible that the sensing electrodes 509 can be connected continuously to the sensor port 519, instead. The electrodes 509 can be types of transducers that can help sense an ECG signal of the patient, e.g. a 12-lead signal, or a signal from a different number of leads, especially if they make good electrical contact with the body of the patient and in particular with the skin of the patient. As with the therapy electrodes 504, 508, the support structure can be configured to be worn by the patient 582 so as to maintain the sensing electrodes 509 on a body of the patient 582. For example, the sensing electrodes 509 can be attached to the inside of the support structure 170 for making good electrical contact with the patient, similarly with the therapy electrodes 504, 508.

Optionally a WMS according to embodiments also includes a fluid that it can deploy automatically between the electrodes and the patient's skin. The fluid can be conductive, such as by including an electrolyte, for establishing a better electrical contact between the electrodes and the skin. Electrically speaking, when the fluid is deployed, the electrical impedance between each electrode and the skin is reduced. Mechanically speaking, the fluid may be in the form of a low-viscosity gel. As such, it will not flow too far away from the location it is released. The fluid can be used for both the therapy electrodes 504, 508, and for the sensing electrodes 509.

The fluid may be initially stored in a fluid reservoir, not shown in FIG. 5. Such a fluid reservoir can be coupled to the support structure. In addition, a WMS according to embodiments further includes a fluid deploying mechanism 574. The fluid deploying mechanism 574 can be configured to cause at least some of the fluid to be released from the reservoir, and be deployed near one or both of the patient body locations to which the therapy electrodes 504, 508 are configured to be attached to the patient's body. In some embodiments, the fluid deploying mechanism 574 is activated prior to the electrical discharge responsive to receiving an activation signal AS from the processor 530, which is described more fully later in this document.

In some embodiments, the unit 500 also includes a measurement circuit 520, as one or more of its modules working together with its sensors and/or transducers. The measurement circuit 520 senses one or more electrical physiological signals of the patient from the sensor port 519, if provided. Even if the unit 500 lacks a sensor port, the measurement circuit 520 may optionally obtain physiological signals through the nodes 514, 518 instead, when the therapy electrodes 504, 508 are attached to the patient. In these cases, the input reflects an ECG measurement. The patient parameter can be an ECG, which can be sensed as a voltage difference between electrodes 504, 508. In addition, the patient parameter can be an impedance (IMP. or Z), which can be sensed between the electrodes 504, 508 and/or between the connections of the sensor port 519 considered pairwise as channels. Sensing the impedance can be useful for detecting, among other things, whether these electrodes 504, 508 and/or the sensing electrodes 509 are not making good electrical contact with the patient's body at the time. These patient physiological signals may be sensed when available. The measurement circuit 520 can then render or generate information about them as inputs, data, other signals, etc. As such, the measurement circuit 520 can be configured to render a patient input responsive to a patient parameter sensed by a sensor. In some embodiments, the measurement circuit 520 can be configured to render a patient input, such as values of an ECG signal, responsive to the ECG signal sensed by the ECG sensing electrodes 509. More strictly speaking, the information rendered by the measurement circuit 520 is output from it, but this information can be called an input because it is received as an input by a subsequent stage, device or functionality.

The unit 500 also includes a processor 530. The processor 530 may be implemented in a number of ways. Such ways include, by way of example and not of limitation, digital and/or analog processors such as microprocessors and Digital Signal Processors (DSPs), controllers such as microcontrollers, software running in a machine, programmable circuits such as Field Programmable Gate Arrays (FPGAs), Field-Programmable Analog Arrays (FPAAs), Programmable Logic Devices (PLDs), Application Specific Integrated Circuits (ASICs), any combination of one or more of these, and so on.

The processor 530 may include, or have access to, a non-transitory storage medium, such as a memory 538 whose contents are described more fully later in this document. Such a memory can have a non-volatile component for storage of machine-readable and machine-executable instructions. A set of such instructions can also be called a program. The instructions, which may also be referred to as “software,” generally provide functionality by performing acts, operations and/or methods as may be disclosed herein or understood by one skilled in the art in view of the disclosed embodiments. In some embodiments, and as a matter of convention used herein, instances of the software may be referred to as a “module” and by other similar terms. Generally, a module includes a set of the instructions so as to offer or fulfill a particular functionality. Embodiments of modules and the functionality delivered are not limited by the embodiments described in this document.

The processor 530 can be considered to have a number of modules. One such module can be a detection module 532. The detection module 532 can include a Ventricular Fibrillation (VF) detector. The patient's sensed ECG from measurement circuit 520, which can be available as inputs, data that reflect values, or values of other signals, may be used by the VF detector to determine whether the patient is experiencing VF. Detecting VF is useful, because VF typically results in SCA. The detection module 532 can also include a Ventricular Tachycardia (VT) detector for detecting VT, a Supra Ventricular Tachycardia (SVT) detector for detecting SVT, and so on.

Another such module in processor 530 can be an advice module 534, which generates advice for what to do. The advice can be based on outputs of the detection module 532. There can be many types of advice according to embodiments. In some embodiments, the advice is a shock/no shock determination that processor 530 can make, for example via advice module 534. The shock/no shock determination can be made by executing a stored Shock Advisory Algorithm. A Shock Advisory Algorithm can make a shock/no shock determination from one or more ECG signals that are sensed according to embodiments, and determine whether or not a shock criterion is met. The determination can be made from a rhythm analysis of the sensed ECG signal or otherwise. For example, there can be shock decisions for VF, VT, etc.

In perfect conditions, a very reliable shock/no shock determination can be made from a segment of the sensed ECG signal of the patient. In practice, however, the ECG signal is often corrupted by electrical noise, which makes it difficult to analyze. Too much noise sometimes causes an incorrect detection of a heart arrhythmia, resulting in a false alarm to the patient. Noisy ECG signals may be handled as described in published US patent application No. US 2019/0030351 A1, and No. US 2019/0030352 A1, and which are incorporated herein by reference.

The processor 530 can include additional modules, such as other module 536, for other functions. In addition, if the internal monitoring device 581 is indeed provided, the processor 530 may receive its inputs, etc.

The unit 500 optionally further includes a memory 538, which can work together with the processor 530. The memory 538 may be implemented in a number of ways. Such ways include, by way of example and not of limitation, volatile memories, Nonvolatile Memories (NVM), Read-Only Memories (ROM), Random Access Memories (RAM), magnetic disk storage media, optical storage media, smart cards, flash memory devices, any combination of these, and so on. The memory 538 is thus a non-transitory storage medium. The memory 538, if provided, can include programs for the processor 530, which the processor 530 may be able to read and execute. More particularly, the programs can include sets of instructions in the form of code, which the processor 530 may be able to execute upon reading. Executing is performed by physical manipulations of physical quantities, and may result in functions, operations, processes, acts, actions and/or methods to be performed, and/or the processor 530 to cause other devices or components or blocks to perform such functions, operations, processes, acts, actions and/or methods. The programs can be operational for the inherent needs of the processor 530, and can also include protocols and ways that decisions can be made by the advice module 534. In addition, the memory 538 can store prompts for the user 582, if this user is a local rescuer. Moreover, the memory 538 can store data. This data can include patient data, system data and environmental data, for example as learned by the internal monitoring device 581 and the outside monitoring device 180. The data can be stored in the memory 538 before it is transmitted out of the unit 500, or be stored there after it is received by the unit 500.

The unit 500 can optionally include a communication module 590, for establishing one or more wired or wireless communication links with other devices of other entities, such as a remote assistance center, Emergency Medical Services (EMS), and so on. The communication links can be used to transfer data and commands. The data may be patient data, event information, therapy attempted, CPR performance, system data, environmental data, and so on. For example, the communication module 590 may transmit wirelessly, e.g. on a daily basis, heart rate, respiratory rate, and other vital signs data to a server accessible over the internet, for instance as described in US 20140043149. This data can be analyzed directly by the patient's physician and can also be analyzed automatically by algorithms designed to detect a developing illness and then notify medical personnel via text, email, phone, etc. The module 590 may also include such interconnected sub-components as may be deemed necessary by a person skilled in the art, for example an antenna, portions of a processor, supporting electronics, outlet for a telephone or a network cable, etc.

The unit 500 may also include a power source 540, which is configured to provide electrical charge in the form of a current. To enable portability of the unit 500, the power source 540 typically includes a battery. Such a battery is typically implemented as a battery pack, which can be rechargeable or not. Sometimes a combination is used of rechargeable and non-rechargeable battery packs. An example of a rechargeable battery 540 was a battery 440 of FIG. 4. Other embodiments of the power source 540 can include an AC power override, for where AC power will be available, an energy-storing capacitor, and so on. Appropriate components may be included to provide for charging or replacing the power source 540. In some embodiments, the power source 540 is controlled and/or monitored by the processor 530.

The unit 500 may additionally include an energy storage module 550. The energy storage module 550 can be coupled to receive the electrical charge provided by the power source 540. The energy storage module 550 can be configured to store the electrical charge received by the power source 540. As such, the energy storage module 550 is where some electrical energy can be stored temporarily in the form of an electrical charge, when preparing it for discharge to administer a shock. In embodiments, the module 550 can be charged from the power source 540 to the desired amount of energy, for instance as controlled by the processor 530. In typical implementations, the module 550 includes a capacitor 552, which can be a single capacitor or a system of capacitors, and so on. In some embodiments, the energy storage module 550 includes a device that exhibits high power density, such as an ultracapacitor. As described above, the capacitor 552 can store the energy in the form of an electrical charge, for delivering to the patient.

As mentioned above, the patient is typically shocked when the shock criterion is met. In particular, in some embodiments the processor 530 is configured to determine from the patient input whether or not a shock criterion is met, and cause, responsive to the shock criterion being met, at least some of the electrical charge stored in the module 550 to be discharged via the therapy electrodes 104, 108 through the ambulatory patient 82 while the support structure is worn by the ambulatory patient 82 so as to deliver the shock 111 to the ambulatory patient 82. There can be different types of such shock criteria, as will be understood from the below. Delivering the electrical charge is also known as discharging and shocking the patient.

For causing the discharge, the unit 500 moreover includes a discharge circuit 555. When the decision is to shock, the processor 530 can be configured to control the discharge circuit 555 to discharge through the patient at least some of all of the electrical charge stored in the energy storage module 550, especially in a desired waveform. When the decision is to merely pace, i.e., to deliver pacing pulses, the processor 530 can be configured to cause control the discharge circuit 555 to discharge through the patient at least some of the electrical charge provided by the power source 540. Since pacing requires lesser charge and/or energy than a defibrillation shock, in some embodiments pacing wiring 541 is provided from the power source 540 to the discharge circuit 555. The pacing wiring 541 is shown as two wires that bypass the energy storage module 550, and only go through a current-supplying circuit 558. As such, the energy for the pacing is provided by the power source 540 either via the pacing wiring 541, or through the energy storage module 550. And, in some embodiments where only a pacer is provided, the energy storage module 550 may not be needed if enough pacing current can be provided from the power source 540. Either way, discharging can be to the nodes 514, 518, and from there to the therapy electrodes 504, 508, so as to cause a shock to be delivered to the patient. The circuit 555 can include one or more switches 557. The switches 557 can be made in a number of ways, such as by an H-bridge, and so on. In some embodiments, different ones of the switches 557 may be used for a discharge where a defibrillation shock is caused to be delivered, than for a discharge where the much weaker pacing pulses are caused to be delivered. The circuit 555 could also be thus controlled via the processor 530, and/or the user interface 580.

The pacing capability can be implemented in a number of ways. ECG sensing may be done in the processor, as mentioned elsewhere in this document, or separately, for demand or synchronous pacing. In some embodiments, however, pacing can be asynchronous. Pacing can be software controlled, e.g., by managing the defibrillation path, or a separate pacing therapy circuit (not shown) could be included, which can receive the ECG sensing, via the circuit 520 or otherwise.

A time waveform of the discharge may be controlled by thus controlling discharge circuit 555. The amount of energy of the discharge can be controlled by how much energy storage module has been charged, and also by how long the discharge circuit 555 is controlled to remain open.

The unit 500 can optionally include other components.

In embodiments, the shock/no shock decision can be made from the patient's heart rate and/or the QRS width of the patient's ECG complexes in the patient's ECG signal. Other parameters may also be used, such as information from a patient impedance signal (Z), information from a motion detection signal (MDET) that may evidence a motion of the patient, and so on. Of course, it is desired to measure these parameters as accurately as possible.

ECG signals in a WCD system may include too much electrical noise to be useful. To ameliorate the problem, multiple ECG sensing electrodes 209 are provided, for presenting many options to the processor 530. These options are different vectors for sensing the ECG signal, as described now in more detail.

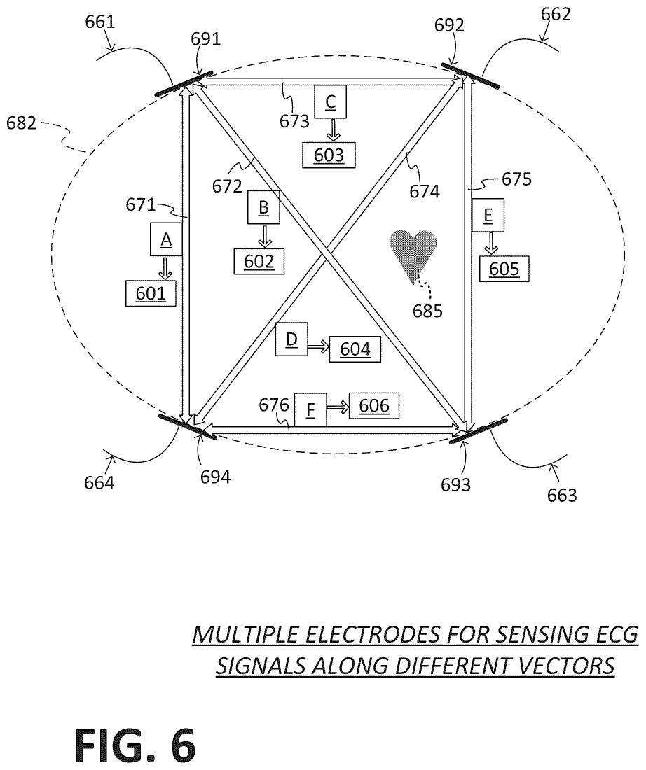

FIG. 6 is a conceptual diagram for illustrating how multiple electrodes of a WCD system may be used for sensing ECG signals along different vectors according to embodiments. A section of a patient 682 having a heart 685 is shown. In FIG. 6, the patient 682 is viewed from the top, the patient 682 is facing downwards, and the plane of FIG. 6 intersects the patient 682 at the torso of the patient.

Four ECG sensing electrodes 691, 692, 693, 694 are maintained on the torso of the patient 682, and have respective wire leads 661, 662, 663, 664. It will be recognized that the electrodes 691, 692, 693, 694 surround the torso, similarly with the four electrodes 209 of FIG. 2A. The ECG electrical potentials that can be measured at the electrodes 691, 692, 693, 694 can have values E1, E2, E3, E4.

Any pair of these four ECG sensing electrodes 691, 692, 693, 694 defines a vector, along which an ECG signal may be sensed and/or measured. As such, the four electrodes 691, 692, 693, 694 pairwise define six vectors 671, 672, 673, 674, 675, 676. FIG. 6 thus illustrates a multi-vector embodiment. Although four electrodes, and thus six vectors, are shown in the example of FIG. 6, other numbers can be implemented. In addition, not all vectors need be considered. For instance, in the example of FIG. 2A, the vectors 673, 676 can be ignored, as they least traverse the torso 682.

In FIG. 6 it will be understood that the electrodes 691, 692, 693, 694 are drawn as if they were on the same plane. This is done because simplicity of explanation is preferred but, strictly speaking, it is not necessarily the case. In fact, the electrodes 691, 692, 693, 694 might not always be on the same plane, in which case the vectors 671, 672, 673, 674, 675, 676 are not necessarily on the same plane, either.

These vectors 671, 672, 673, 674, 675, 676 define channels A, B, C, D, E, F respectively. ECG signals 601, 602, 603, 604, 605, 606 may thus be sensed and/or measured from the channels A, B, C, D, E, F, respectively, and in particular from the appropriate pairings of the wire leads 661, 662, 663, 664 for each channel. The ECG signals 601, 602, 603, 604, 605, 606 may be sensed concurrently or not.

The above-mentioned formalism gives or renders values of the ECG signal that is sensed between pairs of the electrodes. For instance, the ECG signal 601 at channel A has a voltage E1−E2=E12.

It is also possible to use a different formalism that derives ECG signal values for each electrode by itself and at its location, not in a pair with another. This different formalism starts by imagining a point at a virtual position between the 4 electrodes 691, 692, 693, 694, somewhere within the torso of the patient 682. (Such a point is not shown in FIG. 6.) An ECG voltage CM is ascribed to that point. That voltage CM is derived from a statistic of the voltages of at the four electrodes 691, 692, 693, 694. That statistic can be the average. The virtual position continuously changes its virtual position based on the voltages of the four electrodes 691, 692, 693, 694. Of course, there is no actual sensor for sensing the voltage at that point. Nevertheless, this different formalism further imagines a virtual main central terminal (MCT), not shown in FIG. 6, and which is what would be sensing that voltage CM.

In this different formalism, therefore, vectors are considered from each of the four electrodes 691, 692, 693, 694 to the MCT. Their values of their signals, therefore, are considered to be: E1C=E1−CM, E2C=E2−CM, E3C=E3−CM and E4C=E4−CM. In embodiments, the vectors are formed in software by selecting a pair of these signals and subtracting one from the other. So for example, E1C−E2C=(E1−CM)−(E2−CM)=E1−E2+(CM−CM)=E1−E2=E12.

Thus, having multiple channels A, B, C, D, E, F, a WCD may assess which one of them provides the best ECG signal for rhythm analysis and interpretation. Or, instead of just one channel, a WCD may determine that it can keep two or more but not all of the channels and use their ECG signals, for instance as described in U.S. Pat. No. 9,757,581.

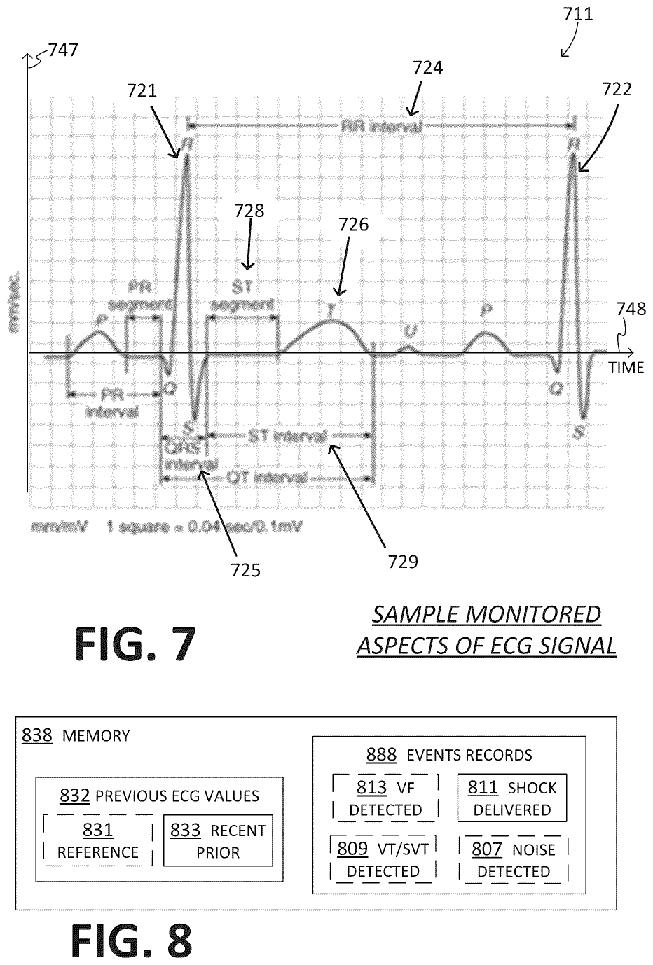

FIG. 7 is a time diagram 711 of a segment of a waveform amplitude of an ideal ECG signal. Actual and non-ideal ECG signal amplitude waveforms may also be shown in other drawings. Even if a patient's signal is almost rarely ideal, FIG. 7 serves well to indicate aspects that can be monitored according to embodiments.

The time diagram 711 shows the ECG amplitude waveform against a vertical axis 747, and over a horizontal time axis 748. Aspects that can be monitored according to embodiments include two QRS peaks 721, 722, an RR interval 724 that is also known as a peak-to-peak interval, a QRS interval 725, a T-wave duration 726, an ST segment duration 728 and an ST interval duration 729. These aspects can have values given, for example, by their intercepts on axes 747, 748. Of course, more than one aspects may be thus monitored according to embodiments, and so on.

FIG. 8 shows a memory 838 that can be as described for the memory 538 of FIG. 5. The memory 838 may store one or more previous ECG values 832. The previous ECG values 832 may include recent prior values for the ECG signal 833, and/or reference values 831 for an ECG signal, such as a QRS template. Such a template may be original, or created during operation from ECG signals of the patient, for instance sensed and captured during times when the patient is determined to have a normal sinus rhythm.

In addition, the processor 530 can cause events records 888 to be stored into the memory 838. Such events records 888 may include a record 813 that reflects that VF was detected, a record 811 that reflects that a shock was delivered, a record 809 that reflects that VT or SVT was detected, a record 807 that reflects that noise was detected, and so on. The events records 807, 809, 813 reflect classifications or decisions that the software has made of the ECG signal sensed as part of the patient input. These events records 888 may also include the date and time, and be examined later by a care giver.

The devices and/or systems mentioned in this document may perform functions, processes, acts, operations, actions and/or methods. These functions, processes, acts, operations, actions and/or methods may be implemented by one or more devices that include logic circuitry. A single such device can be alternately called a computer, and so on. It may be a standalone device or computer, such as a general-purpose computer, or part of a device that has and/or can perform one or more additional functions. The logic circuitry may include a processor and non-transitory computer-readable storage media, such as memories, of the type described elsewhere in this document. Often, for the sake of convenience only, it is preferred to implement and describe a program as various interconnected distinct software modules or features. These, along with data are individually and also collectively known as software. In some instances, software is combined with hardware, in a mix called firmware.

Moreover, methods and algorithms are described below. These methods and algorithms are not necessarily inherently associated with any particular logic device or other apparatus. Rather, they are advantageously implemented by programs for use by a computing machine, such as a general-purpose computer, a special purpose computer, a microprocessor, a processor such as described elsewhere in this document, and so on.

This detailed description may include mind maps, flowcharts, display images, algorithms, and symbolic representations of program operations within at least one computer readable medium. An economy may be achieved in that a single set of flowcharts can be used to describe both programs, and also methods. So, while flowcharts describe methods in terms of boxes, they may also concurrently describe programs.

Methods are now described.

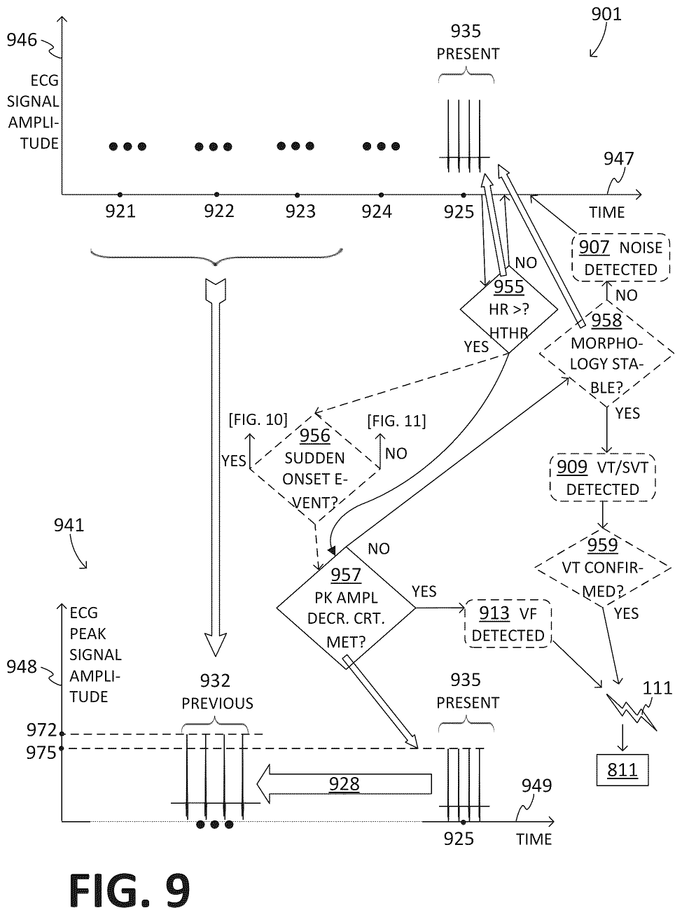

FIG. 9 starts with a diagram 901 at the top, for plotting ECG signal amplitudes. The diagram 901 has a horizontal time axis 947 with time intercepts 921, 922, 923, 924, 925, whose spacings are not necessarily to any scale. The diagram 901 also has a vertical axis 946 for an ECG signal amplitude.

The diagram 901 shows a present ECG signal amplitude 935 at time intercept 925. The diagram 901 does not show, in FIG. 9, any ECG signal amplitudes at the other time intercepts, but such may be presented later in this document. It will be understood that the present ECG signal amplitude 935 is an idealized plot, while a patient's actual ECG signal will likely not appear this way, and especially the ECG signal of a patient who is not having a normal sinus rhythm.

In some embodiments it is determined, from the patient input, whether or not a heart rate (HR) of the patient exceeds a high threshold heart rate (HTHR). The HTHR is a suitable value, for instance 170 beats per minute, indicating a tachycardia that needs to be investigated.

In the diagram 901, this determination is reflected by a decision diamond 955. Right after the time intercept 925, an arrow from the time axis 947 line leads to the decision diamond 955. A near-vertical wide arrow from the decision diamond 955 points to the present ECG signal amplitude 935, from whose values the determination is made.

If the answer at the decision diamond 955 is NO, another arrow points back up to the time axis 947. Then monitoring can be continued at a later time, the operation of the decision diamond 955 can be repeated, and so on.

If the answer at the decision diamond 955 is YES, then according to embodiments, a present ECG peak amplitude may be input, from values for the ECG signal of the patient input. The peak amplitudes are rendered better at a diagram 941 at the bottom of FIG. 9.

The diagram 941 has a horizontal time axis 949, which shares only the time intercept 925 of the time axis 947. The remainder of the time axis 949 is not fully specified in FIG. 9, as indicated by a portion shown only as a dotted line, plus a horizontal dot-dot-dot. This time axis 949 will be specified more in subsequent diagrams. The diagram 941 also has a vertical axis 948 for an ECG peak signal amplitude. In other words, as in the diagram 901, the diagram 941 plots the amplitude evolution of the ECG signal, but the different vertical axis 948 captures the peak values of it. These peak values are typically created by the R peaks of QRS complexes, or by electrical noise.

The diagram 941 repeats the present ECG signal amplitude 935 at the time intercept 925. The peak signal amplitude of the present ECG signal amplitude 935, is measured on the vertical axis 948. This peak is shown by a dot 975, and also by a long horizontal line to the right of the dot 975.

Returning to the decision diamond 955, if the answer is YES then, according to embodiments, a previous ECG peak amplitude is also input, from the one or more previous ECG values 832 stored in the memory 838.

Regarding a previous ECG signal, the diagram 941 also shows a previous ECG signal amplitude 932 that occurs previously to the present ECG signal amplitude 935, as indicated by a lookback arrow 928. It will be observed that the previous ECG signal amplitude 932 is at no specific time intercept. As indicated by the long wide vertical arrow, the previous ECG signal amplitude 932 could be the ECG signal stored in the memory at any one of the time intercepts 921, 922, 923 on the time axis 947, which occur previously to the time intercept 925.

Regarding the previous peak amplitude that is also input, the peak signal amplitude of the previous ECG signal amplitude 932 is measured on the vertical axis 948. The peak amplitude is shown by a dot 972, and by a long horizontal line to the right of the dot 972.

Returning to the decision diamond 955, if the answer is YES then execution may proceed directly to one more decision diamond 957, as seen by a solid curved arrow. Or, execution may optionally proceed first through another optional decision diamond 956, which is described later in this document. Then, after the decision diamond 956, execution may proceed to the decision diamond 957, regardless of what was the answer in the decision diamond 956.

In embodiments, according to the decision diamond 957, it can be determined whether or not a peak amplitude decrease criterion is met. In embodiments, this determination is made from the present ECG peak amplitude, represented by the dot 975 and its horizontal dashed line, and from the previous ECG peak amplitude, represented by the dot 972 and its horizontal dashed line.

In embodiments, the peak amplitude decrease criterion is met responsive to the present ECG peak amplitude being less than the previous ECG peak amplitude by at least a threshold fraction. In this example, the dot 975 is indeed lower than the dot 972, which means that, at the time intercept 925 the ECG peak signal amplitude is lower than it was at the previous time intercept 922. In embodiments, the threshold fraction has a suitable value, for instance 15%.

In embodiments, the peak amplitude decrease criterion serves to detect a shockable heart rhythm of the patient. Indeed, if the answer at the decision diamond 957 is YES then, according to an optional flag 913, VF may be deemed detected. The flag 913 may be implemented in running software. In fact the event 813 may be accordingly recorded in the memory 838.

Then the shock 111 can be administered. In particular, in some embodiments the processor 530 is configured to determine, from the present ECG peak amplitude and from the previous ECG peak amplitude, whether or not a peak amplitude decrease criterion is met and cause, responsive to the peak amplitude decrease criterion being met, at least some of the electrical charge stored in the module 550 to be discharged via the therapy electrodes 104, 108 through the ambulatory patient 82 while the support structure is worn by the ambulatory patient 82 so as to deliver the shock 111 to the ambulatory patient 82.

After the shock 111 of FIG. 9 is administered, the record 811 may be created. In particular, in embodiments, the processor 530 is configured to cause, responsive to causing the discharging 111, the memory 838 to store a record 811 that reflects that a shock 111 was delivered.

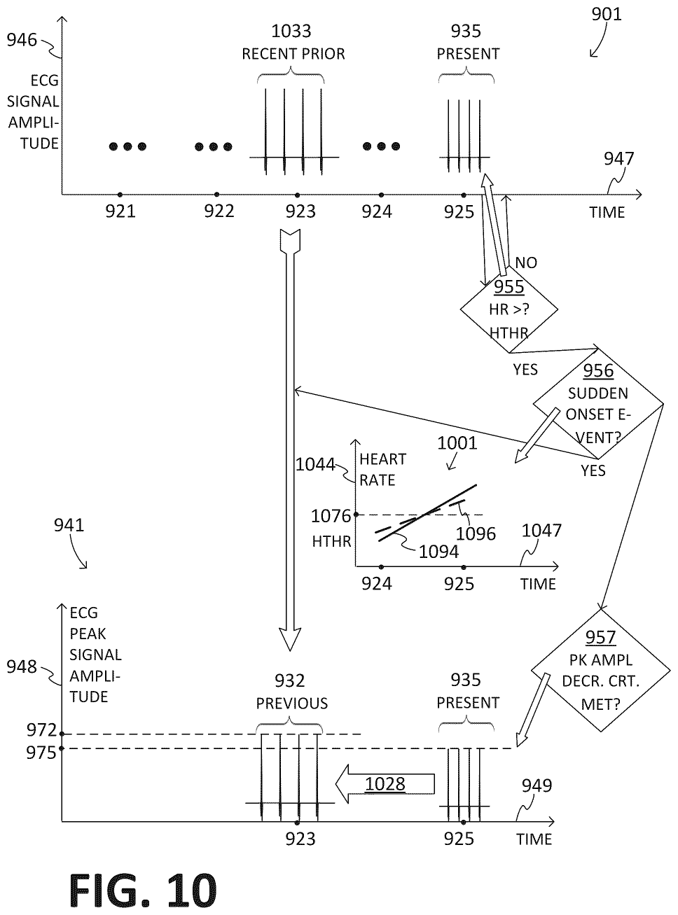

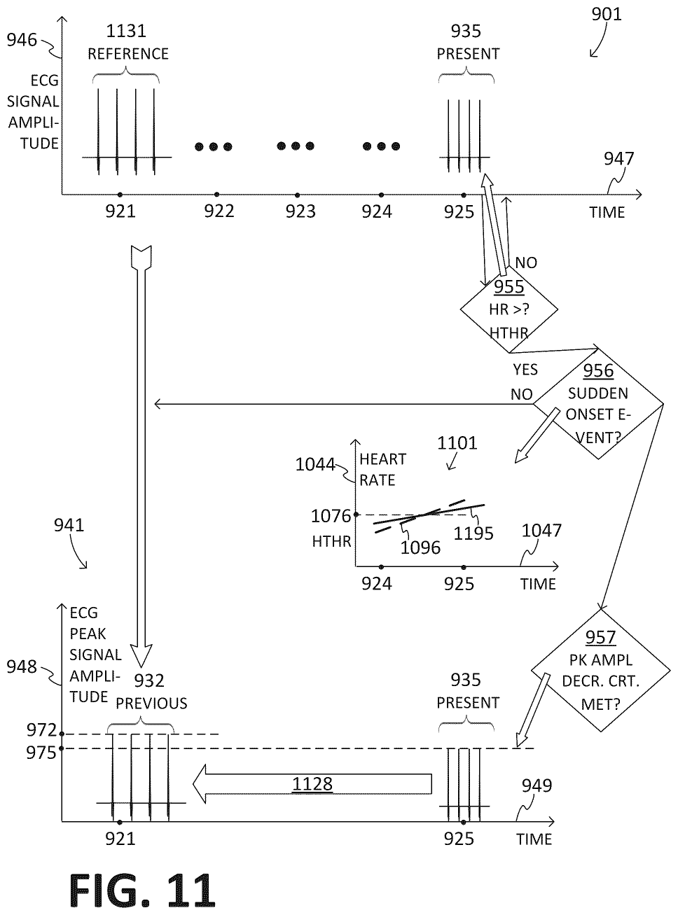

On the other hand, if the answer at the decision diamond 957 is NO, then other operations can take place. These are described later in this document, after the descriptions of FIG. 10 and FIG. 11.

Returning to the time axis 949, the values of the previous ECG signal amplitude 932 may have been stored in the memory at different times, having been obtained at different times. In embodiments, the answer of when the values of the previous ECG signal amplitude 932 were stored in the memory depends on whether or not a sudden onset event was detected. Then the lookback arrow 928 may look back a shorter or longer time.

In particular, in some optional embodiments the processor 530 may detect, from the patient input, whether or not a sudden onset event occurred before it was determined that the heart rate exceeded the high threshold heart rate. Such a sudden onset event can be an event where the heart rate value jumped faster than a jump rate threshold. The jump rate threshold can be a suitable value, such as 20% over 4.8 seconds.

The decision diamond 956 indicates the detection or not of such a sudden onset event. In this example, such an event could have occurred at the time intercept 924 or later, but before the time intercept 925. If the answer at the decision diamond 956 is YES, then one can see more details in FIG. 10. If the answer at the decision diamond 956 is NO, then one can see more details in FIG. 11.

FIG. 10 repeats many elements of FIG. 9. In addition, another diagram 1001 has a horizontal time axis 1047 that replicates a portion of the time axis 947. The diagram 1001 also has a vertical axis 1044 for the detected heart rate. A value 1076 shows the previously mentioned high threshold heart rate (HTHR).

The diagram 1001 plots the detected heart rate as a line 1094. In this example, with the answer of the decision diamond 955 being YES, the line 1094 increased and in fact crossed the HTHR 1076 before the time intercept 925. In fact, this is what was detected, and started the whole process.

The next question is whether or not this increase of the line 1094 qualifies as a sudden onset event. This is effectively the same determination of the decision diamond 956. The answer in this diagram is YES, because, at about that time, the line 1094 has a larger slope than the slope of the jump rate threshold 1096, assuming the increase in the HR has been linear. Here the linearity is for better visualization purposes, when in fact, the behavior of the heart rate (HR) will be more like a jump from one sampling time to another.

In such embodiments, responsive to detecting at the decision diamond 956 that such a sudden onset event occurred, the inputted previous ECG peak amplitude can be determined from the values for the ECG signal that have been stored in the memory less than 30 minutes prior to determining that the heart rate exceeds the high threshold heart rate. The previous ECG signal amplitude 932 could have been sampled, therefore, at the time intercept 923, which is now shown on the time axis 949. Notably, the diagram 941 on FIG. 10 is not inconsistent with the diagram 941 on FIG. 9, because the time axis 949 in FIG. 9 is not specified at that time intercept. Accordingly, the lookback arrow 1028 need not go very far back in time, for instance it can be less than 30 minutes. The corresponding previous ECG signal amplitude 932 of FIG. 9 is seen on the time diagram 901, as a recent prior ECG signal amplitude 1033.

FIG. 11 repeats many elements of FIG. 9 and also of FIG. 10. In fact, a diagram 1101 also replicates many elements of the diagram 1001 of FIG. 10. The diagram 1101 plots the detected heart rate as a line 1195. In this example, with the answer of the decision diamond 955 being YES, the line 1195 increased and in fact crossed the HTHR 1076 before the time intercept 925. Again, this is what started the whole process.

The next question is whether or not this increase of the line 1195 qualifies as a sudden onset event. The answer in this diagram is no because, at about that time, the line 1195 has a smaller slope than the slope of the jump rate threshold 1096.

In such embodiments, responsive to not detecting at the decision diamond 956 that such a sudden onset event occurred, the inputted previous ECG peak amplitude can be determined from the values for the ECG signal that have been stored in the memory longer than 30 minutes prior to determining that the heart rate exceeds the high threshold heart rate. Or, can be less than 30 minutes, even down to one minute or longer, as long as a clean organized low-HR rhythm is available.

The previous ECG signal amplitude 932 could have been sampled, therefore, at the time intercept 921, which is now shown on the time axis 949. Notably, the diagram 941 on FIG. 11 is not inconsistent with the diagram 941 on FIG. 9, because the time axis 949 in FIG. 9 is not specified at that time intercept. Accordingly, the lookback arrow 1128 may go much farther back in time, longer than 30 minutes. The corresponding previous ECG signal amplitude 932 of FIG. 9 is seen on the time diagram 901, as a reference prior ECG signal amplitude 1131. Notably, this reference prior ECG signal amplitude 1131 can be a set of QRS complexes, or even a single QRS complex. In fact, in some embodiments, the processor 530 can be further configured to detect that the patient input has an ECG indicating a normal sinus rhythm, detect an ECG peak amplitude from the patient input when the ECG indicates a normal sinus rhythm, and store a value for the detected ECG peak amplitude in the memory. That can be the peak amplitude whose value is shown by the dot 972, plus the dashed line to the right of the dot 972.

Returning to FIG. 9, if the answer at the decision diamond 957 is NO then, according to additional optional embodiments, the processor 530 may determine whether or not a morphology stability criterion is met by QRS complexes of values for the ECG signal of the patient input. This determination is shown by an optional decision diamond 958, which asks whether a morphology of the present ECG signal amplitude 935 is stable, from one QRS complex to another. In particular, the morphology stability criterion can reflect a similarity of the QRS complexes with respect to each other.

In such embodiments, if the morphology stability criterion is met, then the processor 530 can cause at least some of the stored electrical charge to be thus discharged. In FIG. 9, where the answer to the decision diamond 958 is YES, the arrows lead to the discharge 111. In fact, they may first lead to the optional flag 909, according to which VT or SVT may be detected. And, the event record 809 may be accordingly recorded in the memory 838.

In further optional embodiments, from the flag 909, if specifically VT is detected, the detected VT may be further confirmed before the discharge. This is reflected by an optional decision diamond 959. In particular, responsive to the morphology stability criterion being met, the processor 530 may determine whether or not a VT condition is confirmed. Such a confirmation can be performed, for example, with QRS width criteria, by monitoring the ECG signal for RR interval stability for a preset amount of time, such as 20 sec, 30 sec, or even longer, and morphology comparison to a stored NSR template.

In such embodiments, the processor 530 can cause the at least some of the stored electrical charge to be thus discharged only responsive to the VT condition. Or, the condition may be monitored to see whether it will be reversed on its own, or it will degenerate into VF, for shocking.

However, if the morphology stability criterion is not met, then the processor 530 might not cause any of the stored electrical charge to be thus discharged. In FIG. 9, where the answer to the decision diamond 958 is NO, the arrows lead back to the time axis 947 for further monitoring. In fact, they may first lead to the optional flag 907, according to which noise is detected. And, the event record 807 may be accordingly recorded in the memory 838.

The morphology stability criterion can be met in a number of ways. In some embodiments, the processor 530 is further configured to compute an ECG morphology parameter from the QRS complexes. In such embodiments, the morphology stability criterion can be met responsive to the ECG morphology parameter exceeding a stability threshold. Examples are now described.

In some embodiments, the processor 530 is configured to identify QRS complexes in the values for the ECG signal. In some of these embodiments, the QRS complexes are identified by convolution of a QRS complex with the values for the ECG signal. An example is now described.

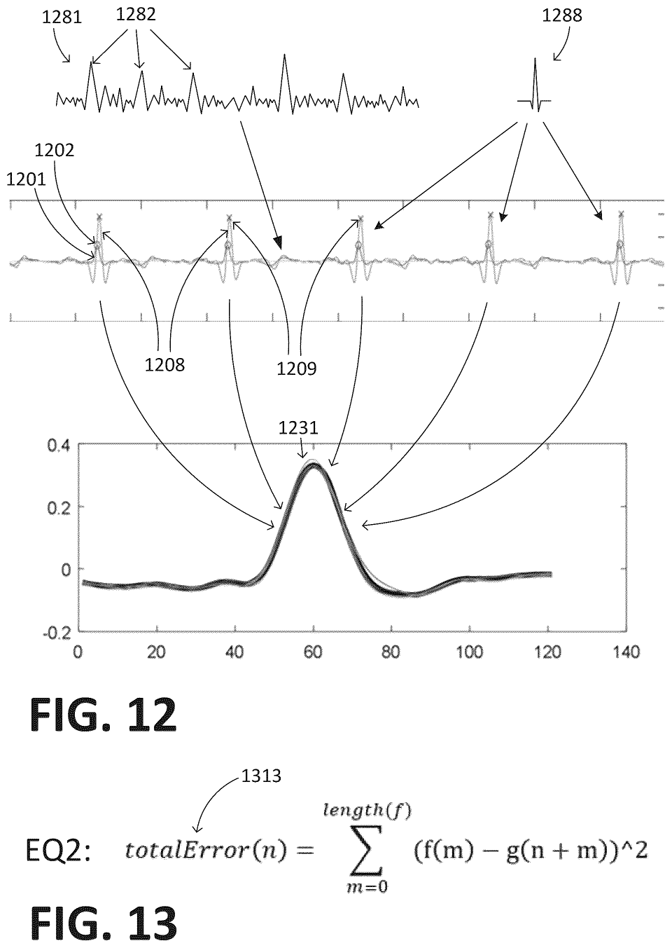

FIG. 12 shows a set of diagrams and waveforms. Waveform 1281 is a sample ECG signal that is shown artificially with much of the high-amplitude noise removed. The waveform 1281 has QRS peaks 1282. A waveform 1288 is a known kernel of a QRS complex, which is used in convolution with an ECG signal waveform, to identify its peaks. The waveform 1288 may have been isolated and catalogued at a much earlier time, for instance when it would be known that the patient is having a normal sinus rhythm.

FIG. 12 also shows a sample ECG signal 1201, which has circled QRS peaks 1202. These QRS peaks 1202 are the ones intended to be detected by the convolution. The result of the convolution is indicated as a convolution waveform 1208 that is superimposed on the ECG signal 1201. The convolution waveform 1208 has peaks 1209 that are marked with “x”s, and which serve as detectors of the QRS peaks 1202, given that the peaks 1209 are known to occur slightly after the QRS peaks 1202.

In such embodiments, the processor 530 is configured to derive a signal-averaged QRS complex from at least some of the identified QRS complexes. The averaging can be to reduce noise and variability. This is shown in FIG. 12 as a signal-averaged QRS complex 1231 being derived from the QRS complexes identified in the diagram just above. Indeed, such QRS complexes can be clipped out of the signal and overlaid in a table, which is why they are all shown superimposed, and together with their signal-averaged QRS complex 1231. The ECG signal may have been filtered, for deriving the signal-averaged QRS complex 1231.

In such embodiments, then, the processor 530 is configured to compute individual statistics of differences of the signal-averaged QRS complex from the identified QRS complexes. In such embodiments, then, the ECG morphology parameter can be computed from the individual statistics. In fact, it can be computed from groups of peak values of the individual statistics. Given that the signal-averaged QRS complex was derived from the identified QRS complexes themselves, these individual statistics are a measure of the self-similarity of the identified QRS complexes. An example is now described.

In such embodiments, the inventor uses the name “QRS Organization” to measure the self-similarity of the identified QRS complexes. The inventor further uses additional names for additional quantities. A process can be as follows:
A) Start with an average QRS complex, such as the signal-averaged QRS complex 1231.
B) Slide the average QRS complex along the ECG signal.
C) For every possible alignment of the two signals, compute the squared difference. It will be recognized that these are examples of the above-mentioned statistics of differences.
D) Compute the sum of the squared differences. In this example, the result is a signal that the inventor calls “totalError”, and which is indicated in FIG. 13 as a quantity 1313.
D) Take the −log10 of totalError and subtract the mean value. This is referred to as “goodness.”
E) High pass filter the goodness to produce a filtered Goodness.
F) Identify peaks of the filtered Goodness. For instance, find the maximum value within a window and slide the window across the signal in 25 sample increments. The array of peak values is referred to as the peak Goodness.

The value of the Organization can range, theoretically, between 2 for perfect organization and 0 for no organization. Two extreme examples are now described.

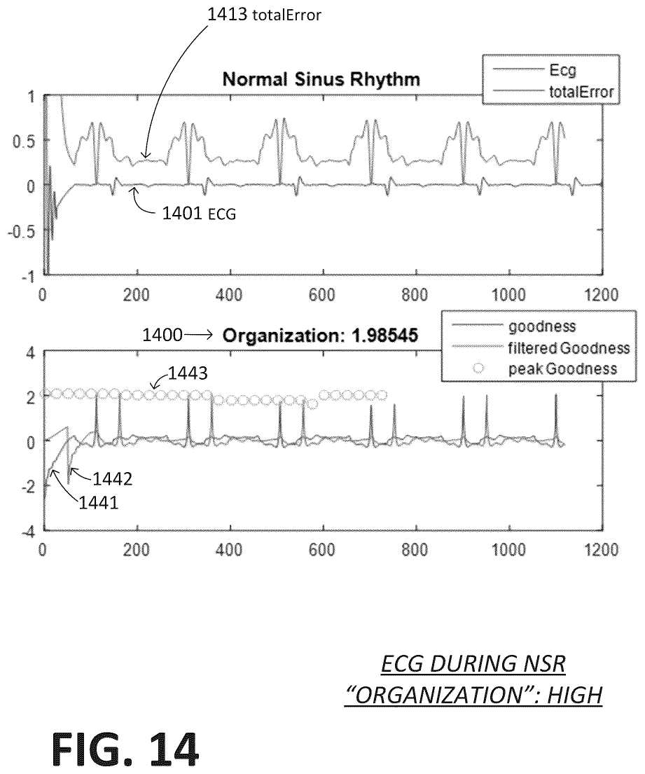

As a first example, FIG. 14 has an upper panel and a lower panel. The upper panel shows a sample ECG signal 1401 during a heart's normal sinus rhythm (NSR). The totalError 1413 is computed according to the equation FIG. 13, as if it were a signal. The totalError 1413 goes to near zero for every QRS complex. It is not lined up with the ECG signal because no effort is made to compensate for the kernel length.

The lower panel shows the goodness as a line 1441, the filtered goodness as a line 1442, and the values of the peak goodness as circles 1443. The resulting value for Organization 1400 is 1.98, which indicates a relatively organized signal, which in turn indicates a clean signal with consistent morphology.

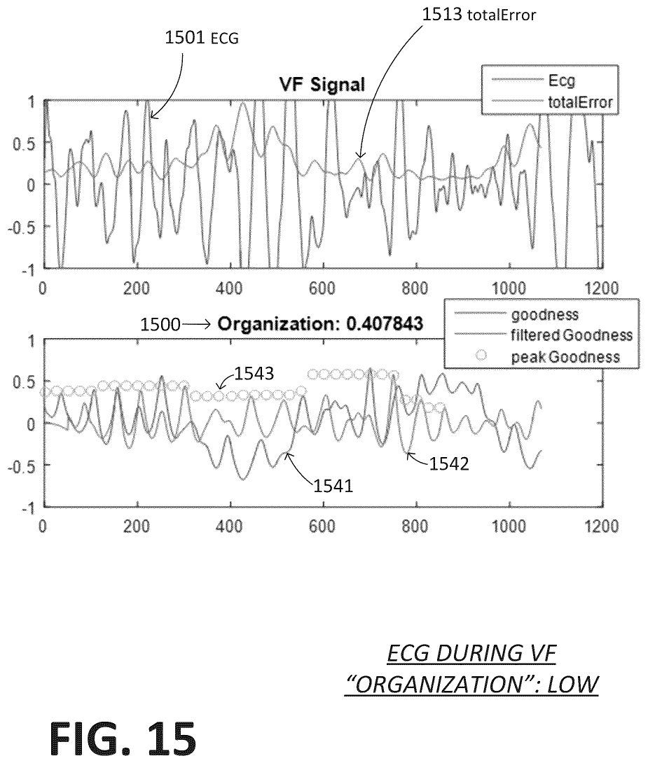

As a second example, FIG. 15 has an upper panel and a lower panel. The upper panel shows a sample ECG signal 1501 during a heart's VF episode. The totalError 1513 is computed according to the equation FIG. 13, as if it were a signal. The totalError 1513 does not approach to zero as close as the totalError 1413 did.

The lower panel shows the goodness as a line 1541, the filtered goodness as a line 1542, and the values of the peak goodness 1543 as circles. The peak goodness values 1543 do not go as high as the example of FIG. 14. The resulting value for Organization 1500 is 0.4, which indicates a rather disorganized signal, which in turn indicates either VF or noise.

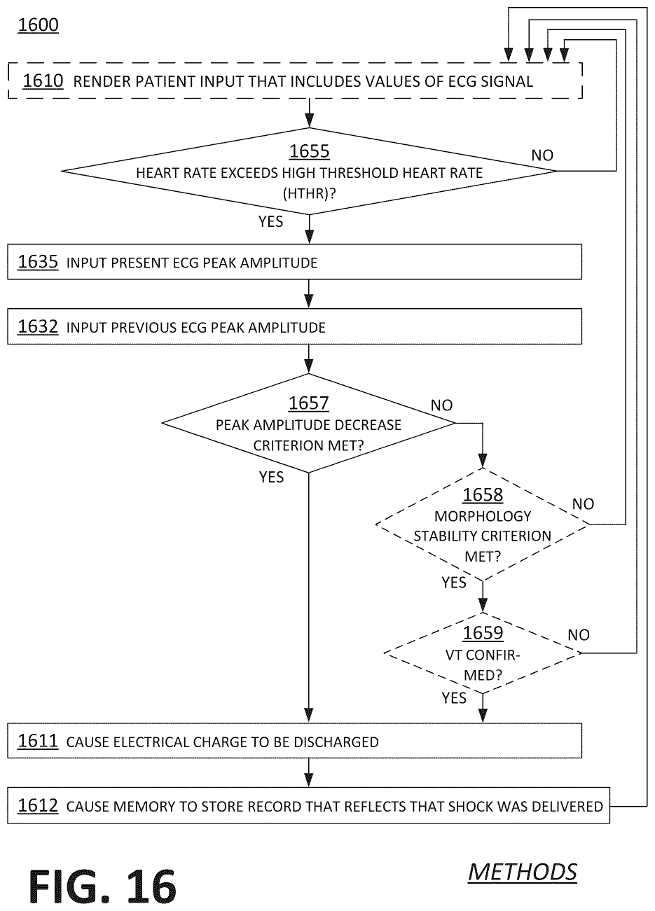

FIG. 16 shows a flowchart 1600 for describing methods according to embodiments.

According to an optional operation 1610, a patient input is rendered that includes values for the ECG signal. The operation 1610 may be performed by a measurement circuit and responsive to a sensed ECG signal.

According to another operation 1655, it may be determined, from the patient input, whether or not a heart rate of the patient exceeds a high threshold heart rate (HTHR). If NO, then execution may return to the operation 1610.

If YES then, according to another operation 1635, a present ECG peak amplitude may be input. The operation 1635 may be performed responsive to the heart rate exceeding the high threshold heart rate and from the values for the ECG signal of the patient input.

According to another operation 1632, a previous ECG peak amplitude may be input from one or more previous ECG values stored in a memory. The operation 1632 may be performed responsive to the heart rate exceeding the high threshold heart rate. In some embodiments, it may be further detected, from the patient input, whether or not a sudden onset event occurred before it was determined that the heart rate exceeds the high threshold heart rate. In such embodiments, this may affect what is the previous ECG peak amplitude that is input, for instance as seen in the contrast between FIG. 10 and FIG. 11.

According to another operation 1657 it may be determined, from the present ECG peak amplitude and from the previous ECG peak amplitude, whether or not a peak amplitude decrease criterion is met.

If YES then, according to another operation 1611, at least some of stored electrical charge may be caused to be discharged via the therapy electrode through the ambulatory patient while the support structure is worn by the ambulatory patient so as to deliver a shock to the ambulatory patient. The operation 1611 may be performed responsive to the peak amplitude decrease criterion being met.

According to another operation 1612, a memory can be caused to store a record that reflects that a shock was delivered. The operation 1611 may be performed responsive to the discharging being caused, per the operation 1611.

If at the operation 1657 the answer is NO then, according to another, optional operation 1658, it may be determined whether or not a morphology stability criterion is met by QRS complexes of values for the ECG signal of the patient input. The operation 1658 may be performed responsive to the peak amplitude decrease criterion not being met. The morphology stability criterion may reflect a similarity of the QRS complexes with respect to each other. In some embodiments, an ECG morphology parameter may be computed from the QRS complexes. In such embodiments, the morphology stability criterion can be met responsive to the ECG morphology parameter exceeding a stability threshold. If at the operation 1658 the answer is NO, then execution may return to the operation 1610.

If at the operation 1658 the answer is YES, then a VT condition or an SVT condition may have been detected. Then according to another, optional operation 1659, if a VT condition has been detected, it may be confirmed. If NO, then execution may return to the operation 1610, and there is no discharge. If YES, then execution may proceed again to the operation 1611.

### FURTHER EMBODIMENTS

In some embodiments, the ECG signal is monitored, and a shock decision is made where needed. In such embodiments, the shock decision may nevertheless be canceled, depending on other conditions being met. Examples are now described.

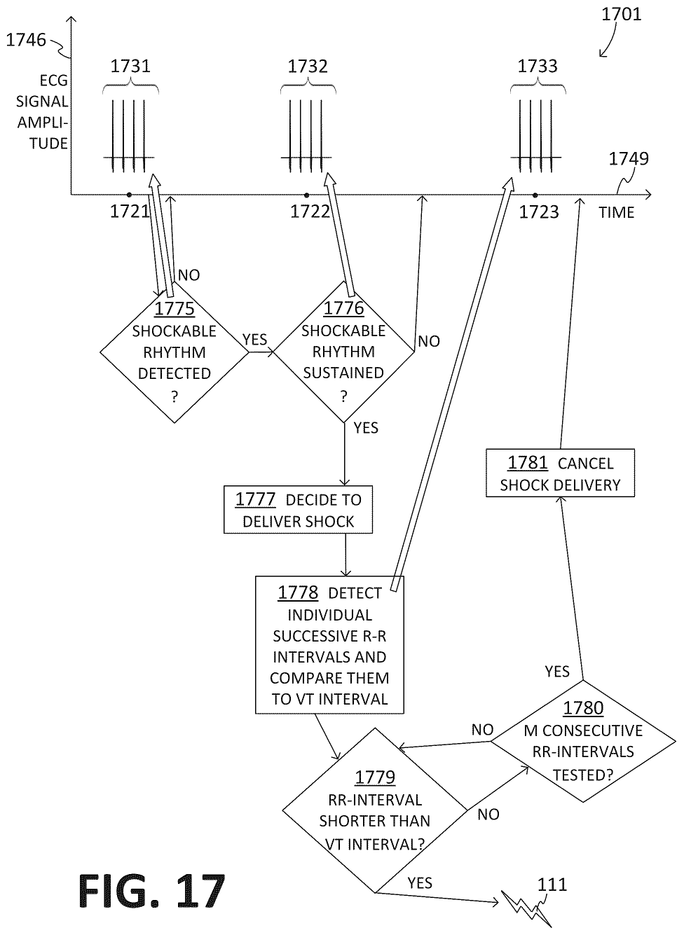

FIG. 17 starts with a diagram 1701 at the top. The diagram 1701 has a horizontal time axis 1749 with time intercepts 1721, 1722, 1723. The diagram 1701 also has a vertical axis 1746 for an ECG signal amplitude. The diagram 1701 shows iconic groups of ECG signal amplitudes 1731, 1732 1733, at the time intercepts 1721, 1722, 1723, respectively. Like with time diagram 901, it will be understood that the ECG signal amplitudes in diagram 1701 are idealized plots.

Right after the time intercept 1721, an arrow leads from the time axis 1749 to a decision diamond 1775, where it is inquired whether or not a shockable rhythm is detected in the group of ECG signal amplitudes 1731. This inquiry is also indicated by a near-vertical wide arrow that points from the decision diamond 1775 up to the ECG signal amplitudes 1731. This inquiry may be performed in many ways, as is known in the art. If the answer is NO, then the mind map of FIG. 17 returns with another arrow to the time axis 1749, and the process may be repeated for the next ECG signal amplitudes.

If at the decision diamond 1775 the answer is YES then, according to another decision diamond 1776, it is inquired whether or not the detected shockable rhythm is sustained for a preset amount of time. This is also indicated by a near-vertical wide arrow that points from the decision diamond 1776 up to the group of ECG signal amplitudes 1732, which follow the ECG signal amplitudes 1731. If the answer is NO, then the mind map of FIG. 17 returns with another arrow to the time axis 1749, and these processes may be repeated for the next group of ECG signal amplitudes.

If at the decision diamond 1776 the answer is YES then, according to an operation 1777, a decision is made to implement shock delivery. The decision is made in software. In some embodiments, it may be even followed-up in hardware, for instance the capacitor 552 may start to be charged. In this example, the decision is made after the group of ECG signal amplitudes 1732 has been fully detected.

Then, according to an operation 1778, individual successive R-R intervals are detected. A sample R-R interval 724 is described earlier in this document. The detection is also indicated by a near-vertical wide arrow that points from the operation 1778 up to the next available group of ECG signal amplitudes 1733. While the whole group is shown as pointed to, in fact the detection (operation 1778) and subsequent checking (operation 1779) are for the R-R intervals one-by-one.

As the detection of the operation 1778 is taking place, at another decision diamond 1779 it is inquired whether or not any RR interval is detected to be shorter than the VT interval. This is also called checking the RR interval. The individual R-R intervals can be checked for how long they are, and compared to a threshold time duration. The threshold time duration can be a suitable duration, such as the VT interval. The VT interval is the duration, in time, of the inverse of a VT rate threshold. For example, if the VT rate threshold is 170 bpm, the VT interval in seconds is 60/170. If at the decision diamond 1779 the answer is YES, then the shock 111 can be administered. If, at the decision diamond 1779, the answer is NO then, at another decision diamond 1780 it is inquired whether or not a number M of consecutive RR intervals have been thus tested, all of which therefore would be longer than the threshold time duration. The number M can be a suitable number, such as five. If, at the decision diamond 1780, the answer is NO then execution returns to the decision diamond 1779 for another RR interval.

If at the decision diamond 1780 the answer is YES then, according to another operation 1781, the decision to deliver a shock is canceled. After that, the mind map of FIG. 17 returns with another arrow to the time axis 1749, and these process may be repeated for the next ECG signal amplitudes. In other words, after the shock delivery decision at the operation 1777, a shock is delivered only when the RR interval from the previous R-wave is shorter than VT interval within M R-R intervals.

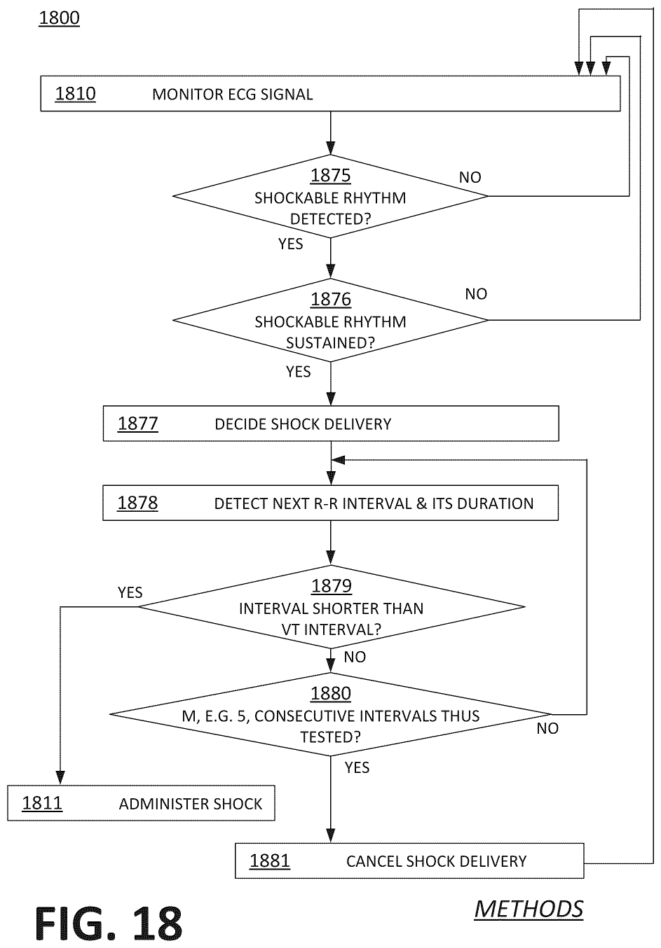

FIG. 18 shows a flowchart 1800 for describing methods according to embodiments. According to an operation 1810, an ECG signal may be monitored.

According to another operation 1875, it may be determined whether or not a shockable rhythm is detected by the operation 1810. This may be performed as described above with reference to the decision diamond 1775. If the answer is NO, then execution may return to the operation 1810.

If at the operation 1875 the answer is YES then, according to another operation 1876, it may be determined whether or not the detected shockable rhythm is sustained for a preset amount of time. This may be performed as described above with reference to the decision diamond 1776. If the answer is NO, then execution may return to the operation 1810.

If at the operation 1876 the answer is YES then, according to another operation 1877, a decision may be made to deliver a shock. This may be performed as described above with reference to the operation 1777.

Then, according to another operation 1878, a next R-R interval may be detected, along with its duration. The duration can be compared to a suitable duration, such as the VT interval. This operation 1878 may be performed as described above with reference to the operation 1778.

According to another operation 1879, it may be determined whether or not the duration of the detected RR interval is shorter than the threshold time duration. This may be performed as described above with reference to the decision diamond 1779. If at the operation 1879 the answer is YES then, according to another operation 1811, a shock can be administered.

If, at the operation 1879, the answer is YES then, according to another operation 1880, it is inquired whether or not a number M of consecutive RR intervals have been thus tested, all of which therefore would be longer than the threshold time duration. This may be performed as described above with reference to the decision diamond 1780. If the answer is NO then execution may return to the operation 1878, to test the next RR interval.

If, at the operation 1880, the answer is YES then, according to another operation 1881, the shock delivery that was decided upon in the operation 1877 can be canceled. This operation 1881 may be performed as described above with reference to the operation 1781. Then execution may return to the operation 1810.

In the methods described above, each operation can be performed as an affirmative act or operation of doing, or causing to happen, what is written that can take place. Such doing or causing to happen can be by the whole system or device, or just one or more components of it. It will be recognized that the methods and the operations may be implemented in a number of ways, including using systems, devices and implementations described above. In addition, the order of operations is not constrained to what is shown, and different orders may be possible according to different embodiments. Examples of such alternate orderings may include overlapping, interleaved, interrupted, reordered, incremental, preparatory, supplemental, simultaneous, reverse, or other variant orderings, unless context dictates otherwise. Moreover, in certain embodiments, new operations may be added, or individual operations may be modified or deleted. The added operations can be, for example, from what is mentioned while primarily describing a different system, apparatus, device or method.

At least one of the methods of this description, when implemented by a computer, can be performed differently at the rate of at least 10 times per second.

A person skilled in the art will be able to practice the present invention in view of this description, which is to be taken as a whole. Details have been included to provide a thorough understanding. In other instances, well-known aspects have not been described, in order to not obscure unnecessarily this description.

Some technologies or techniques described in this document may be known. Even then, however, it does not necessarily follow that it is known to apply such technologies or techniques as described in this document, or for the purposes described in this document.

This description includes one or more examples, but this fact does not limit how the invention may be practiced. Indeed, examples, instances, versions or embodiments of the invention may be practiced according to what is described, or yet differently, and also in conjunction with other present or future technologies. Other such embodiments include combinations and sub-combinations of features described herein, including for example, embodiments that are equivalent to the following: providing or applying a feature in a different order than in a described embodiment; extracting an individual feature from one embodiment and inserting such feature into another embodiment; removing one or more features from an embodiment; or both removing a feature from an embodiment and adding a feature extracted from another embodiment, while providing the features incorporated in such combinations and sub-combinations.

In general, the present disclosure reflects preferred embodiments of the invention. The attentive reader will note, however, that some aspects of the disclosed embodiments extend beyond the scope of the claims. To the respect that the disclosed embodiments indeed extend beyond the scope of the claims, the disclosed embodiments are to be considered supplementary background information and do not constitute definitions of the claimed invention.

In this document, the phrases “constructed to”, “adapted to” and/or “configured to” denote one or more actual states of construction, adaptation and/or configuration that is fundamentally tied to physical characteristics of the element or feature preceding these phrases and, as such, reach well beyond merely describing an intended use. Any such elements or features can be implemented in a number of ways, as will be apparent to a person skilled in the art after reviewing the present disclosure, beyond any examples shown in this document.

Incorporation by reference: References and citations to other documents, such as patents, patent applications, patent publications, journals, books, papers, web contents, have been made throughout this disclosure. All such documents are hereby incorporated herein by reference in their entirety for all purposes.

Parent patent applications: Any and all parent, grandparent, great-grandparent, etc. patent applications, whether mentioned in this document or in an Application Data Sheet (“ADS”) of this patent application, are hereby incorporated by reference herein as originally disclosed, including any priority claims made in those applications and any material incorporated by reference, to the extent such subject matter is not inconsistent herewith or prohibited by law.

Reference numerals: In this description a single reference numeral may be used consistently to denote a single item, aspect, component, or process. Moreover, a further effort may have been made in the preparation of this description to use similar though not identical reference numerals to denote other versions or embodiments of an item, aspect, element, component or process that are identical, or at least similar or related. Where made, such a further effort was not required, but was nevertheless made gratuitously so as to facilitate comprehension by the reader. Even where made in this document, such a further effort might not have been made completely consistently for all of the versions or embodiments that are made possible by this description. Accordingly, the description controls in defining an item, aspect, element, component or process, rather than its reference numeral. Any similarity in reference numerals may be used to infer a similarity in the text, but not to confuse aspects where the text or other context indicates otherwise.

The claims of this document define certain combinations and sub-combinations of elements, features and acts or operations, which are regarded as novel and non-obvious. The claims also include elements, features and acts or operations that are equivalent to what is explicitly mentioned. Additional claims for other such combinations and sub-combinations may be presented in this or a related document. These claims are intended to encompass within their scope all changes and modifications that are within the true spirit and scope of the subject matter described herein. The terms used herein, including in the claims, are generally intended as “open” terms. For example, the term “including” means “including but not limited to,” the term “having” means “having at least,” etc. If a specific number is ascribed to a claim recitation, this number is a minimum but not a maximum unless stated otherwise. As used throughout this document, reference to a singular includes the plural. For example, where a claim recites “a” component or “an” item, it means that the claim can have one or more of that component or that item. In addition, use of terms which are inherently singular (e.g., datum, criterion, agendum, etc.) include their plural forms (e.g., data, criteria, agenda, etc.) unless specifically excluded. In other words, “datum” means “datum or data”. “criterion” means “criterion or criteria”. and the like.

In this document, and especially in the appended claims, if the word “when” is used in combination with an identified event, the word “when” is being used to indicate that the event is a trigger and it is not used in a temporal sense. For example, the phrase “when an event occurs, performing a step” as used in this document means that the occurrence of the event is a trigger to performing the step, but the step need not necessarily be performed in any temporal relationship with the event.

In construing the claims of this document, the inventor(s) invoke 35 U.S.C. § 112(f) only when the words “means for” or “steps for” are expressly used in the claims. Accordingly, if these words are not used in a claim, then that claim is not intended to be construed by the inventor(s) in accordance with 35 U.S.C. § 112(f).

1. A wearable medical system (“WMS”) for a patient, the WMS including at least:
a support structure configured to be worn by the patient;
sensing electrodes configured to sense electrical activity of the heart of the patient and output an Electrocardiogram (ECG) signal;
an energy storage module;
a therapy electrode coupled to the energy storage module;
a memory; and
one or more processors configured to:
store, in the memory, ECG values based on the ECG signal,
generate a patient input based on the ECG values,
determine, from the patient input, whether a heart rate of the patient exceeds a high threshold heart rate,
when the heart rate exceeds the high threshold heart rate, determine:
a present ECG peak amplitude from the ECG signal,
input a previous ECG peak amplitude from ECG values stored in the memory,
determine, from the present ECG peak amplitude and from the previous ECG peak amplitude, whether a peak amplitude decrease criterion is met,
cause, responsive at least in part to a determination that the peak amplitude decrease criterion is met, electrical charge to be discharged from the energy storage device via the therapy electrode through the patient so as to deliver a shock to the patient; and
store in the memory a record that indicates the shock was delivered.

2. The WMS of claim 1, in which:
the high threshold heart rate is about 170 beats per minute.

3. The WMS of claim 1, in which:
the peak amplitude decrease criterion comprises the present ECG peak amplitude being less than the previous ECG peak amplitude by at least a threshold fraction.

4. The WMS of claim 3, in which:
the threshold fraction is about 15%.

5. The WMS of claim 3, in which the processor is further configured to:
detect, from the patient input, whether a sudden onset event occurred before a determination that the heart rate exceeded the high threshold heart rate, and
in which, responsive to a detection of such a sudden onset event, the previous ECG peak amplitude is determined from the ECG values stored in the memory fewer than 30 minutes prior to the determination that the heart rate exceeds the high threshold heart rate.

6. The WMS of claim 5, in which:
the detection of the sudden onset event includes determining that the heart rate jumped faster than a jump rate threshold.

7. The WMS of claim 6, in which:
the jump rate threshold is about 20% over about 4.8 seconds.

8. The WMS of claim 3, in which the processor is further configured to:
detect, from the patient input, whether a sudden onset event occurred before a determination that the heart rate exceeded the high threshold heart rate, and
when the sudden onset event is not detected, the previous ECG peak amplitude is determined from the ECG values stored in the memory at least 30 minutes prior to the determination that the heart rate exceeds the high threshold heart rate.

9. The WMS of claim 8, in which:
the detection of the sudden onset event includes determining that the heart rate jumped faster than a jump rate threshold.

10. The WMS of claim 9, in which:
the jump rate threshold is about 20% over about 4.8 seconds.

11. The WMS of claim 8, in which the processor is further configured to:
detect that the patient input has an ECG indicating a normal sinus rhythm;
detect an ECG peak amplitude from the patient input when the ECG indicates the normal sinus rhythm; and
store a value for the detected ECG peak amplitude in the memory.

12. The WMS of claim 1, in which the processor is further configured to:
determine, responsive to the peak amplitude decrease criterion not being met, whether a morphology stability criterion is met by QRS complexes of the ECG values, the morphology stability criterion indicative of a similarity of the QRS complexes with respect to each other; and
cause, responsive at least in part to a determination that the morphology stability criterion is met, electrical charge to be discharged from the energy storage device.

13. The WMS of claim 12, in which the processor is further configured to:
determine, responsive to the morphology stability criterion being met, whether a Ventricular Tachycardia (VT) condition is confirmed; and
cause electrical charge to be discharged from the energy storage device responsive at least in part to a determination that the VT condition is confirmed.

14. The WMS of claim 12, in which the processor is further configured to:
compute an ECG morphology parameter from the QRS complexes, wherein the morphology stability criterion is met when the ECG morphology parameter exceeds a stability threshold.

15. The WMS of claim 14, in which the processor is further configured to:
identify QRS complexes in the ECG values;
derive a signal-averaged QRS complex from at least some of the identified QRS complexes; and
compute individual statistics of differences of the signal-averaged QRS complex from the identified QRS complexes; and
in which the ECG morphology parameter is computed from the individual statistics.

16. The WMS of claim 15, in which:
the QRS complexes are identified by convolution of a QRS complex with the ECG values.

17. The WMS of claim 15, in which:
the ECG morphology parameter is computed from groups of peak values of the individual statistics.

18.-51. (canceled)
